SEQUENCE LISTING (48 sequences)

SEQ ID NO: 1 - protein, 759 aa, Artificial Sequence
  multi-span CAR protein based on the multi-span transmembrane protein CD20, CD20-CAR-TM-1
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGSTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 2 - protein, 764 aa, Artificial Sequence
  multi-span CAR protein based on the multi-span transmembrane protein CD20, CD20-CAR-TM-2
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 3 - protein, 686 aa, Artificial Sequence
  multi-span CAR protein based on the multi-span transmembrane protein CD20, CD20-CAR-TM-3
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 27 aa, Artificial Sequence
  transmembrane domain
FWVLVVVGGVLACYSLLVTVAFIIFWV

SEQ ID NO: 5 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 6 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 7 - protein, 20 aa, Artificial Sequence
  spacer sequence, human IgG1 hinge
AEPKSPDKTHTCPPCPKDPK

SEQ ID NO: 8 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 9 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 10 - protein, 112 aa, Artificial Sequence
  CD3 Z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 37 aa, Artificial Sequence
  CD28 endodomain
KRSRLLHSDYMNMTPRRPGPTRKHYQPYAPPRDFAAY

SEQ ID NO: 12 - protein, 37 aa, Artificial Sequence
  OX40 endodomain
RRDQRLPPDAHKPPGGGSFRTPIQEEQADAHSTLAKI

SEQ ID NO: 13 - protein, 42 aa, Artificial Sequence
  41BB endodomain
KRGRKKLLYIFKQPFMRPVQTTQEEDGCSCRFPEEEEGGCEL

SEQ ID NO: 14 - protein, 152 aa, Artificial Sequence
  CD28 and CD3 Zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 188 aa, Artificial Sequence
  CD28, OX40 and CD3 Zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 191 aa, Artificial Sequence
  41BB, OX40 and CD3 Zeta endodomains
KRGRKKLLYIFKQPFMRPVQTTQEEDGCSCRFPEEEEGGCELRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 21 aa, Artificial Sequence
  signal peptide
MGTSLLCWMALCLLGADHADG

SEQ ID NO: 18 - protein, 21 aa, Artificial Sequence
  signal peptide
MSLPVTALLLPLALLLHAARP

SEQ ID NO: 19 - protein, 20 aa, Artificial Sequence
  signal peptide
MAVPTQVLGLLLLWLTDARC

SEQ ID NO: 20 - protein, 20 aa, Artificial Sequence
  cleavage site, 2A-like sequence
RAEGRGSLLTCGDVEENPGP

SEQ ID NO: 21 - protein, 402 aa, Artificial Sequence
  Inducible Caspase 9 (iCasp9) sequence
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 22 - protein, 157 aa, Artificial Sequence
  RQR8 sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 23 - protein, 23 aa, Artificial Sequence
  CD20 transmembrane domain
VQIMNGLFHIALGGLLMIPAGIY

SEQ ID NO: 24 - protein, 23 aa, Artificial Sequence
  CD20 transmembrane domain
VTVWYPLWGGIMYIISGSLLAAT

SEQ ID NO: 25 - protein, 22 aa, Artificial Sequence
  CD20 transmembrane domain
MNSLSLFAAISGMILSIMDILN

SEQ ID NO: 26 - protein, 23 aa, Artificial Sequence
  CD20 transmembrane domain
FLGILSVMLIFAFFQELVIAGIV

SEQ ID NO: 27 - protein, 19 aa, Artificial Sequence
  CD28 transmembrane domain
IWAPLAGTCGVLLLSLVIT

SEQ ID NO: 28 - protein, 22 aa, Artificial Sequence
  CD53 transmembrane domain
YVLFFFNLLFWICGCCILGFGI

SEQ ID NO: 29 - protein, 15 aa, Artificial Sequence
  CD53 transmembrane domain
VFVIVGSIIMVVAFL

SEQ ID NO: 30 - protein, 26 aa, Artificial Sequence
  CD53 transmembrane domain
LLMSFFILLLIILLAEVTLAILLFVY

SEQ ID NO: 31 - protein, 26 aa, Artificial Sequence
  CD53 transmembrane domain
NFLYIGIITICVCVIEVLGMSFALTL

SEQ ID NO: 32 - protein, 23 aa, Artificial Sequence
  CD81 transmembrane domain
KYLLFVFNFVFWLAGGVILGVAL

SEQ ID NO: 33 - protein, 24 aa, Artificial Sequence
  CD81 transmembrane domain
GIYILIAVGAVMMFVGFLGCYGAI

SEQ ID NO: 34 - protein, 23 aa, Artificial Sequence
  CD81 transmembrane domain
LLGTFFTCLVILFACEVAAGIWG

SEQ ID NO: 35 - protein, 24 aa, Artificial Sequence
  CD81 transmembrane domain
KLYLIGIAAIVVAVIMIFEMILSM

SEQ ID NO: 36 - protein, 22 aa, Artificial Sequence
  CD82 transmembrane domain
YFLFLFNLIFFILGAVILGFGV

SEQ ID NO: 37 - protein, 19 aa, Artificial Sequence
  CD82 transmembrane domain
MGAYVFIGVGAVTMLMGFL

SEQ ID NO: 38 - protein, 27 aa, Artificial Sequence
  CD82 transmembrane domain
LLGLYFAFLLLILIAQVTAGALFYFNM

SEQ ID NO: 39 - protein, 22 aa, Artificial Sequence
  CD82 transmembrane domain
GIILGVGVGVAIIELLGMVLSI

SEQ ID NO: 40 - protein, 698 aa, Artificial Sequence
  multi-span transmembrane protein AU29657
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSMGMSSLKLLKYVLFFFNLLFWICGCCILGFGIYLLIHNNFGVLFHNLPSLTLGNVFVIVGSIIMVVAFLGCMRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGSGSIKENKCLLMSFFILLLIILLAEVTLAILLFVYEQKLNEYVAKGLTDSIHRYHSDNSTKAAWDSIQSFLQCCGINGTSDWTSGPPASCPSDRKVEGCYAKARLWFHSNFLYIGIITICVCVIEVLGMSFALTLNCQIDKTSQTIGL

SEQ ID NO: 41 - protein, 677 aa, Artificial Sequence
  multi-span transmembrane protein AU29658
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSMGMSSLKLLKYVLFFFNLLFWICGCCILGFGIYLLIHNNFGVLFHNLPSLTLGNVFVIVGSIIMVVAFLGCMGSIKENKCLLMSFFILLLIILLAEVTLAILLFVYEQKLNEYVAKGLTDSIHRYHSDNSTKAAWDSIQSFLQCCGINGTSDWTSGPPASCPSDRKVEGCYAKARLWFHSNFLYIGIITICVCVIEVLGMSFALRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 715 aa, Artificial Sequence
  multi-span transmembrane protein AU29659
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSMGVEGCTKCIKYLLFVFNFVFWLAGGVILGVALWLRHDPQTTNLLYLELGDKPAPNTFYVGIYILIAVGAVMMFVGFLGCYGAIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGSQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 43 - protein, 699 aa, Artificial Sequence
  multi-span transmembrane protein AU29660
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 708 aa, Artificial Sequence
  multi-span transmembrane protein AU29661
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSMGSACIKVTKYFLFLFNLIFFILGAVILGFGVWILADKSSFISVLQTSSSSLRMGAYVFIGVGAVTMLMGFLGCIGARVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGSQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 45 - protein, 725 aa, Artificial Sequence
  multi-span transmembrane protein AU29662
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCNHRNRRRVCKCPRPVVSGGGGSMGSACIKVTKYFLFLFNLIFFILGAVILGFGVWILADKSSFISVLQTSSSSLRMGAYVFIGVGAVTMLMGFLGCIGAVNEVRCLLGLYFAFLLLILIAQVTAGALFYFNMGKLKQEMGGIVTELIRDYNSSREDSLQDAWDYVQAQVKCCGWVSFYNWTDNAELMNRPEVTYPCSCEVKGEEDNSLSVRKGFCEAPGNRTQSGNHPEDWPVYQEGCMEKVQAWLQENLGIILGVGVGVAIIELLGMVLSICLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 789 aa, Artificial Sequence
  multi-span transmembrane protein AU29896
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSGGGGSTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATRKRGRKKLLYIFKQPFMRPVQTTQEEDGCSCRFPEEEEGGCELLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 788 aa, Artificial Sequence
  multi-span transmembrane protein AU29897
METDTLLLWVLLLWVPGSTGDIQMTQTTSSLSASLGDRVTISCRASQDISKYLNWYQQKPDGTVKLLIYHTSRLHGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSSGGGGSTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATRKRGRKKLLYIFKQPFMRPVQTTQEEDGCSCRFPEEEEGGCELLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 154 aa, Artificial Sequence
  41BB CD3 Zeta endodomains
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2018/0371056 A1
(43) Pub. Date: Dec. 27, 2018

(54) Multi-Span Chimeric Antigen Receptor

(71) Applicant: UCL Business PLC, London (GB)

(72) Inventors: Martin Pulé, London (GB); Weng-Kit Cheung, London (GB)

(73) Assignee: UCL Business PLC, London (GB)

(21) Appl. No.: 15/775,541

(22) PCT Filed: Nov. 11, 2016
(86) PCT No.: PCT/GB2016/053544

§ 371 Date: May 11, 2018

(30) Foreign Application Priority Data
Nov. 11, 2015 (GB) 1519900.3

(51) Int. Cl. C07K 14/705; A61K 35/17; C12N 5/0783; C07K 16/28; C07K 14/725

(52) U.S. Cl. CPC C07K 14/70596; A61K 35/17; C12N 5/0636; C07K 16/2803; C07K 14/70517; A61K 38/00; C07K 2319/03; C07K 2319/02; C07K 2319/10; C07K 2317/622; C07K 14/7051

(57) ABSTRACT

The present invention relates to a multi-span chimeric antigen receptor (CAR) which comprises: i) at least one extra-cellular antigen binding domain; ii) a plurality of linked transmembrane domains; and iii) at least one intracellular signalling domain.

### FIELD OF THE INVENTION

The present invention relates to a chimeric antigen receptor.

### BACKGROUND TO THE INVENTION

Traditionally, antigen-specific T-cells have been generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem as transgenic expression of Chimeric Antigen Receptor (CAR) allows generation of large numbers of T-cells specific to any surface antigen by ex vivo viral vector transduction of a bulk population of peripheral blood T-cells.

CARs are proteins which graft the specificity of an antigen binder, such as a monoclonal antibody (mAb), to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common forms of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Current designs of CARs are type I transmembrane proteins comprising an extracellular antigen binding domain and an intracellular signal transmitting endodomain. To enable more than one signal to be transmitted, multiple constituent signalling endodomains are typically attached together in a compound intracellular signalling domain. This design has some limitations: firstly, steric hindrance of second messenger molecules may occur since the different constituent signalling endodomains are close together. Secondly, it is difficult to easily incorporate more than three constituent signalling endodomains effectively. Thirdly, only a single antigen binding domain is typically accommodated in a classical CAR architecture.

Optimal CAR signalling is essential to enable engraftment and expansion of CAR T-cells. Clinical data suggests that engraftment and expansion of CAR T-cells which results in their prolonged persistence is important for durable responses. However, excessively potent signaling can result in immune activation syndromes. Further, if CAR T-cells successfully engraft, since only one antigen is targeted, relapse of the cancer in a cognate antigen negative form can occur.

Physiologically, optimal T-cell persistence and function requires an optimal set of signals to be provided at particular levels to allow physiological function which results in a controlled deletion of the infected tissue compartment and survival of a proportion of specific T-cells to enter immunological memory. A typical immune response targets more than a single antigen. Traditional CARs may not provide the most optimal arrangement for optimal signalling and targeting.

There is thus a need for alternative CARs that are not associated with the disadvantages and problems mentioned above.

### SUMMARY OF ASPECTS OF THE INVENTION

The present inventors have developed multi-span CARs which comprise a plurality of linked transmembrane domains. The use of a multi-span CAR allows multiple antigen-binding domains and/or multiple intracellular signalling domains to be included in the CAR. There are several potential advantages to this architecture, as described herein.

Thus in a first aspect the present invention relates to a multi-span CAR which comprises: i) at least one extracellular antigen binding domain; ii) a plurality of linked transmembrane domains; and iii) at least one intracellular signalling domain.

The CAR may comprise more than one antigen-binding domain. Each antigen binding domain may be located at a different extracellular domain of the multi-span transmembrane protein. Each antigen binding domain may recognise a different antigen.

The multi-span CAR may comprise more than one intracellular signalling domain. Each intracellular signalling domain may be located at a different intracellular domain of the multi-span CAR. Each intracellular signalling domain may comprise a different signalling endodomain(s). Alternatively, each intracellular signalling domain may comprise the same signalling endodomain(s).

The intracellular signalling domain may comprise at least one of CD3 zeta endodomain, CD28 endodomain, 41BB endodomain,OX40 endodomain, CD2 endodomain, Inducible T-cell costimulator (ICOS) endodomain, CD27 endodomain, BTLA endodomain, CD30 endodomain, GITR endodomain or HVEM endodomain. The intracellular signalling domain may comprise at least one of CD3 zeta endodomain, CD28 endodomain, 41BB endodomain and OX40 endodomain.

The intracellular signalling domain may comprise a single endodomain selected from CD3 zeta endodomain, CD28 endodomain, 41BB endodomain,OX40 endodomain, CD2 endodomain, Inducible T-cell costimulator (ICOS) endodomain, CD27 endodomain, BTLA endodomain, CD30 endodomain, GITR endodomain or HVEM endodomain.

The intracellular signalling domain may comprise CD3 zeta endodomain, CD28 endodomain and 41BB endodomain or CD3 zeta endodomain, CD28 endodomain and OX40 endodomain.

The multi-span CAR may comprise one or more transmembrane domains of CD20, CD53, CD80 or CD81 or a variant of one or more transmembrane domains of CD20, CD53, CD80 or CD81 having at least 80% sequence identity thereto.

The multi-span CAR may comprise one or more transmembrane domains of CD20 or variants thereof having at least 80% sequence identity.

In a further aspect the present invention provides a nucleic acid encoding a multi-span CAR according to the present invention.

In another aspect the present invention provides a vector comprising a nucleic acid sequence according to the present invention.

In a further aspect the present invention provides a cell which expresses a multi-span CAR according to the present invention. The cell may be a T cell, NK cell, γδ T cell, myeloid cell or macrophage. In one embodiment the cell is a T cell or NK cell.

In a further aspect the present invention provides a pharmaceutical composition comprising a cell according to the present invention and a pharmaceutically acceptable carrier, diluent or excipient.

In another aspect the present invention provides pharmaceutical composition according to the present invention for use in treating and/or preventing a disease.

In another aspect the present invention relates to a method for treating and/or preventing a disease which comprises the step of administering a pharmaceutical composition according to the present invention to a subject.

The method may comprise the following steps:
(i) isolation of a cell containing sample from a subject;
(ii) transduction or transfection of the cell with a nucleic acid or a vector according to the present invention; and
(iii) administering the cell from (ii) to the subject.

In a further aspect the present invention provides the use of a nucleic, a vector or a cell according to the present invention in the manufacture of a medicament for the treatment and/or prevention of a disease.

The disease may be cancer.

The present invention also relates to a method for making a cell according to the present invention, which comprises the step of introducing: a nucleic acid or a vector according to the present invention into the cell.

The cell may be from a sample isolated from a subject.

### DESCRIPTION OF THE FIGURES

FIG. 1—a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2—Schematic diagram of an illustrative multi-span CAR. (a) The multi-span CAR is based on CD20 (b). In construct EN-1, the CD19 scFv fmc63 was cloned in frame with the stalk, transmembrane domain and polar anchor from CD8 (labelled as STK), with CD3-Zeta endodomain shown as a red bar. CD3-Zeta endodomain was cloned in frame so that this largely replaced the first intracellular domain of CD20. (c) In construct EN-2, the scFv-CD8 fusion was connected to a truncated amino-terminus of CD20 and CD3-Zeta endodomain was inserted into the 2nd intracelluar domain of CD20. (d) In construct EN-3, the scFv-CD8 fusion was connected to a truncated amino-terminus of CD20 and the CD3-Zeta endodomain replaced the 3rd intraceullar domain.

FIG. 3—a) control 1st generation CAR and the three multi-span CARs (EN-1, EN-2 and EN-3) were cloned into a bicistronic retroviral vector where RQR8 was co-expressed with the CAR via a foot-and-mouth 2A-like sequence. (b) BW5 T-cells were transduced with the different vectors and stained with the monoclonal antibody QBEnD10 (which recognizes RQR8), and recombinant CD19-Fc fusion (which recognizes the CAR). Non-transduced cells, control CAR transduced cells and EN-1 EN-2 and EN-3 transduced cells were analyzed. Dot-plot of QBEnD10 vs CD19-Fc staining is shown. (c) The BW5 T-cells were incubated with SupT1 cells (which are CD19 negative), and SupT1.CD19 cells (which are SupT1 cells engineered to express CD19). IL-2 secretion after this co-culture was measured by ELISA.

FIG. 4—Schematic diagram illustrating different combinations of signaling domains which may be utilised in a multi-span CAR. (a) In this design, CD28, 41BB and CD3-Zeta endodomains are each expressed on a different intracellular domains of the multi-span protein. (b) In this design, each intracellular domain has a compound intracellular signaling domain consisting of CD28, 41BB and Zeta endodomains. (c) In this design, each intracellular domain has one 41BB endodomain, but only one contains a CD3-Zeta component.

FIG. 5—Schematic diagram showing a multi-span CAR with multiple targeting domains. (a) a multi-span CAR with a single targeting domain; (b) a multi-span CAR with two targeting domains, each on a different extracellular domain.

FIG. 6—Sequences of the illustrative multi-span CAR proteins based on the multi-span transmembrane protein CD20 and other multi-span transmembrane proteins

FIG. 7—Multi-span CAR derived from CD53. (a) The fmc63 scFv attached to the CD8-stalk and CD8-transmembrane domain were fused to CD53. The endodomain of CD3Zeta was either inserted into the central intracellular loop or the carboxy-terminus of CD53. BW5 T-cells were transduced with these constructs as well as a standard CAR as described by Campana (Imai et al.; Leuk. Off. J. Leuk. Soc. Am. Leuk. Res. Fund UK 18, 676-684 (2004)). (b) T-cells were then challenged by either SupT1 cells or SupT1 cells engineered to express CD19 and IL2 secretion measured.

FIG. 8—Multi-span CAR derived from CD81. (a) The fmc63 scFv attached to the CD8-stalk and CD8-transmembrane domain were fused to CD81. The endodomain of CD3Zeta was either inserted into the central intracellular loop or the carboxy-terminus of CD81. BW5 T-cells were transduced with these constructs as well as a standard CAR as described by Campana (as above). (b) T-cells were then challenged by either SupT1 cells or SupT1 cells engineered to express CD19 and IL2 secretion measured.

FIG. 9—Multi-span CAR derived from CD82. (a) The fmc63 scFv attached to the CD8-stalk and CD8-transmembrane domain were fused to CD83. The endodomain of CD3Zeta was either inserted into the central intracellular loop or the carboxy-terminus of CD81. BW5 T-cells were transduced with these constructs as well as a standard CAR as described by Campana (as above). (b) T-cells were then challenged by either SupT1 cells or SupT1 cells engineered to express CD19 and IL2 secretion measured.

FIG. 10—Multi-span 41BB-Zeta CAR. (a) A standard Campana CAR (left) was compared with a multi-span CAR based on CD20 with 41BB in the first and second intracellular domain and 41BB-Zeta in the 3^{rd} intracellular domain. (b) Primary human T-cells transduced with these two constructs were challenged with SupT1 cells and SupT1 cells expressing CD19 at 1:4 T-cell to target ratio for 24 hours and killing efficiency measured by flow-cytometry.

FIG. 11—Multi-span CD28-41BB-Zeta CAR. (a) A standard Campana CAR (left) was compared with a multi-span CAR based on CD20 with CD28 in the first intracellular domain, 41BB in the second intracellular domain and 41BB-Zeta in the 3rd intracellular domain. (b) Primary human T-cells transduced with these two constructs were labelled with CFSE and challenged with SupT1 cells and SupT1 cells expressing CD19 at 1:4 T-cell to target ratio for 5 days and proliferation determined by CFSE dilution

### DETAILED DESCRIPTION OF THE INVENTION

Chimeric Antigen Receptors (CARs)

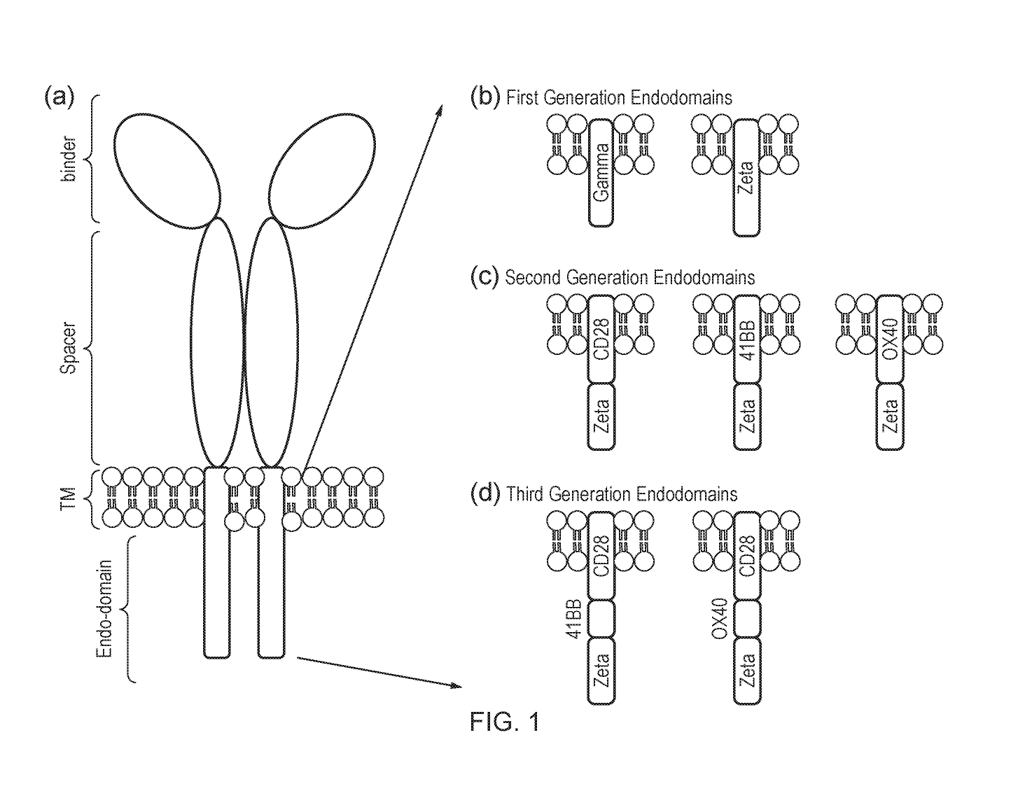

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like or ligand-based antigen binding site. A spacer domain may be necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

The present invention provides a multi-span CAR which comprises: i) at least one extracellular antigen binding domain; ii) a plurality of linked transmembrane domains; and iii) at least one intracellular signalling domain.

The use of a multi-span CAR allows multiple antigen-binding domains and/or multiple intracellular signalling domains to be included in the CAR. There are several advantages provided by the multi-span architecture. Firstly, individual signalling endodomains may be positioned independently of one another (i.e. located at different intracellular domains of the multi-span CAR)—which avoids potential problems with steric hindrance of secondary messengers which may be associated with classical CAR architecture. This independent arrangement means that each individual signalling endodomain is positioned proximal to the membrane—such positioning is typically optimal for signalling functions. Further, it is possible to include more constituent signalling endodomains per antigen-binding domain than is typically possible with a classical CAR. This is typically not possible with a classical CAR architecture as the intracellular domain of the classical CAR becomes too large and too crowded, and the most distal signalling endodomains become too far removed from the membrane for efficient or productive signalling. It is also possible to alter the ratio between different antigen binding domains and different signalling endodomains (e.g. by including multiple intracellular signalling domains in the multi-span CAR—with different constituent signalling endodomains in different intracellular signalling domains).

A multi-span CAR of the present invention may also allow more convenient targeting of more than one antigen compared to a classical CAR architecture. This may be achieved by including more than one antigen-binding domain in the multi-span CAR. Typically, each antigen binding domain will be located at a different extracellular domain of the multi-span CAR.

Transmembrane Domain

The multi-span CAR of the present invention comprises a plurality of linked transmembrane domains.

Each native transmembrane protein adopts a particular orientation in the membrane. This reflects both the asymmetric manner in which it is synthesized and inserted into the lipid bilayer in the endoplasmic reticulum and the different functions of its cytosolic and extracellular domains. These domains are separated by the membrane-spanning segments of the polypeptide chain, which contact the hydrophobic environment of the lipid bilayer and are composed largely of amino acid residues with nonpolar side chains. Because the peptide bonds themselves are polar and because water is absent, all peptide bonds in the bilayer are driven to form hydrogen bonds with one another. The hydrogen bonding between peptide bonds is maximized if the polypeptide chain forms a regular alpha helix as it crosses the bilayer, and this is how the great majority of the membrane-spanning segments of polypeptide chains are thought to traverse the bilayer.

A multi-span transmembrane protein (also referred to as a multi-pass transmembrane protein) comprises a plurality of linked transmembrane domains such that the polypeptide chain traverses the membrane multiple times. Accordingly, a multi-span CAR comprises a polypeptide chain which traverses the membrane multiple times. Thus, a multi-span CAR comprises a plurality of transmembrane domains, with adjacent transmembrane domains linked/connected by extracellular or intracellular amino acid loops. Each transmembrane domain typically comprises a hydrophobic alpha helix or a beta sheet.

Suitably, a plurality may mean that the multi-span CAR comprises at least two, at least three, at least four, at least five, at least six, at least seven, at least eight, at least nine or at least ten transmembrane domains.

Suitably, the multi-span CAR may comprise from two up to ten, two up to eight, four up to eight or four up to six transmembrane domains.

Suitably, the multi-span CAR may comprise two, three, four, five, six, seven, eight, nine or ten transmembrane domains. Suitably, the multi-span CAR may comprise five transmembrane domains.

The multi-span CAR may have any suitable conformation and/or orientation in the cell membrane when expressed in a cell.

The multi-span CAR may have one or both termini on the extracellular side of the cell membrane when expressed in a cell. The multi-span CAR may have one or more amino acid loop linkers on the extracellular side of the cell membrane when expressed in a cell.

The multi-span CAR may have one or both termini on the intracellular side of the cell membrane when expressed in a cell. The multi-span CAR may have one or more amino acid loop linkers on the intracellular side of the cell membrane when expressed in a cell.

The multi-span CAR may have both termini located on the intracellular side of the cell membrane when expressed in a cell (i.e. a Type IV-A transmembrane protein conformation). The multi-span CAR may have one terminus located on the intracellular side of the cell membrane and one terminus located on the extracellular side of the cell membrane when expressed in a cell (i.e. a Type IV-B transmembrane protein conformation).

The transmembrane domain may be any protein structure which is thermodynamically stable in a membrane. This is typically an alpha helix comprising of several hydrophobic residues. The transmembrane domain of any transmembrane protein can be used to supply a transmembrane portion. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (see U.S. Pat. No. 7,052,906 B1 which describes transmembrane components and is incorporated herein by reference).

The transmembrane domain(s) may comprise a hydrophobic alpha helix. The transmembrane domain(s) may be derived from CD28.

The transmembrane domain of CD28 is shown as SEQ ID NO: 27 (IWAPLAGTCGVLLLSLVIT). The transmembrane domain(s) may comprise the sequence shown as SEQ ID NO: 27 or a variant thereof having at least 80, 85, 90, 95, 98 or 99% sequence identity with SEQ ID NO: 27, provided that the variant sequence retains the capacity to traverse the cell membrane.

SEQ ID NO: 4
FWVLVVVGGVLACYSLLVTVAFIIFWV

The variant may have at least 80, 85, 90, 95, 98 or 99% sequence identity with SEQ ID NO: 4, provided that the variant sequence retains the capacity to traverse the cell membrane.

The multi-span CAR may comprise one or more transmembrane domains from a native multi-span transmembrane protein. A large number of multi-span transmembrane proteins are known in the art. By way of example, human multi-span transmembrane proteins are summarised as part of a review of human transmembrane proteins by Schiöth et al. (BMC Biology; 2009; 7:50). Multi-span transmembrane proteins suitable for use in a multi-span CAR of the present invention include, but are not limited to, MS4A family proteins, G-protein coupled receptors, class B scavenger receptors, ion channels, claudins, gap junction proteins, tretraspanins, TMEM16, TM9SF, TMEM63, IFITM, synaptogyrins and synaptophysins.

The multi-span transmembrane protein may comprise at least two, at least three, at least four, at least five, at least six, at least seven, at least eight, at least nine or at least ten transmembrane domains.

The multi-span transmembrane protein may comprise two, three, four, five, six, seven, eight, nine or ten transmembrane domains.

The multi-span transmembrane protein may be a member of the MS4A family. The MS4A (membrane-spanning 4-domain family, subfamily A) family of proteins contains well-known members including MS4A1 (CD20), MS4A2 (FcεRIβ) and MS4A3 (HTm4). Members of this nascent protein family are characterized by common structural features and similar intron/exon splice boundaries and display unique expression patterns among hematopoietic cells and nonlymphoid tissues.

The multi-span transmembrane protein may be CD20, CD53, CD81 or CD82.

The multi-span transmembrane protein may be CD20. An example CD20 protein is the human CD20 protein having the UniProtKB accession number P11836. This exemplified sequence is 297 amino acids in length. CD20 is an activated-glycosylated phosphoprotein with four transmembrane domains, two extracellular domains and three intracellular domains. It is expressed on the surface of all B-cells beginning at the pro-B phase (CD45R+, CD117+) and its concentration progressively increases on the cell membrane until maturity.

The multi-span CAR may comprise one or more of the transmembrane domains derived from a transmembrane domain of CD20. The multi-span CAR may comprise at least two or at least three of the transmembrane domains from CD20. The multi-span CAR may comprise one, two, three or all four of the transmembrane domains from CD20.

The multi-span CAR may comprise more than one copy of one, two, three or four of the transmembrane domains from CD20, each transmembrane domain from CD20 forming a separate transmembrane domain in the multi-span CAR.

The transmembrane domains of CD20 are shown as SEQ ID NO: 23-26.

SEQ ID NO: 23
VQIMNGLFHIALGGLLMIPAGIY
SEQ ID NO: 24
VTVWYPLWGGIMYIISGSLLAAT
SEQ ID NO: 25
MNSLSLFAAISGMILSIMDILN
SEQ ID NO: 26
FLGILSVMLIFAFFQELVIAGIV

The transmembrane domain may be a variant of a transmembrane domain from CD20. The variant may have at least 80, 85, 90, 95, 98 or 99% sequence identity to SEQ ID NO: 23, 24, 25 or 26, provided that the variant sequence retains the capacity to traverse the membrane.

The multi-span transmembrane protein may be CD53. An example CD53 protein is the human CD20 protein having the UniProtKB accession number P19397. This exemplified sequence is 219 amino acids in length, including the initiator methionine. CD53 is a member of the transmembrane 4 superfamily and is a cell surface glycoprotein that is known to complex with integrins. It contributes to the transduction of CD2-generated signals in T cells and natural killer cells and has been suggested to play a role in growth regulation.

The multi-span CAR may comprise one or more of the transmembrane domains derived from a transmembrane domain of CD53. The multi-span CAR may comprise at least two or at least three of the transmembrane domains from CD53. The multi-span CAR may comprise one, two, three or all four of the transmembrane domains from CD53.

The multi-span CAR may comprise more than one copy of one, two, three or four of the transmembrane domains from CD53, each transmembrane domain from CD53 forming a separate transmembrane domain in the multi-span CAR.

The transmembrane domains of CD53 are shown as SEQ ID NO: 28-31.

SEQ ID NO: 28-
YVLFFFNLLFWICGCCILGFGI
SEQ ID NO: 29-
VFVIVGSIIMVVAFL
SEQ ID NO: 30-
LLMSFFILLLIILLAEVTLAILLFVY
SEQ ID NO: 31-
NFLYIGIITICVCVIEVLGMSFALTL

The transmembrane domain may be a variant of a transmembrane domain from CD53. The variant may have at least 80, 85, 90, 95, 98 or 99% sequence identity to SEQ ID NO: 28, 29, 30 or 31, provided that the variant sequence retains the capacity to traverse the membrane.

The multi-span transmembrane protein may be CD81. An example CD81 protein is the human CD20 protein having the UniProtKB accession number P60033. This exemplified sequence is 236 amino acids in length.

The multi-span CAR may comprise one or more of the transmembrane domains derived from a transmembrane domain of CD81. The multi-span CAR may comprise at least two or at least three of the transmembrane domains from CD81. The multi-span CAR may comprise one, two, three or all four of the transmembrane domains from CD81.

The multi-span CAR may comprise more than one copy of one, two, three or four of the transmembrane domains from CD81, each transmembrane domain from CD81 forming a separate transmembrane domain in the multi-span CAR.

The transmembrane domains of CD81 are shown as SEQ ID NO: 32-35.

SEQ ID NO: 32-
KYLLFVFNFVFWLAGGVILGVAL
SEQ ID NO: 33-
GIYILIAVGAVMMFVGFLGCYGAI
SEQ ID NO: 34-
LLGTFFTCLVILFACEVAAGIWG
SEQ ID NO: 35-
KLYLIGIAAIVVAVIMIFEMILSM

The transmembrane domain may be a variant of a transmembrane domain from CD81. The variant may have at least 80, 85, 90, 95, 98 or 99% sequence identity to SEQ ID NO: 32, 33, 34 or 35, provided that the variant sequence retains the capacity to traverse the membrane.

The multi-span transmembrane protein may be CD82. An example CD82 protein is the human CD20 protein having the UniProtKB accession number P27701. This exemplified sequence is 267 amino acids in length.

The multi-span CAR may comprise one or more of the transmembrane domains derived from a transmembrane domain of CD82. The multi-span CAR may comprise at least two or at least three of the transmembrane domains from CD82. The multi-span CAR may comprise one, two, three or all four of the transmembrane domains from CD82.

The multi-span CAR may comprise more than one copy of one, two, three or four of the transmembrane domains from CD82, each transmembrane domain from CD82 forming a separate transmembrane domain in the multi-span CAR.

The transmembrane domains of CD82 are shown as SEQ ID NO: 36-39.

SEQ ID NO: 36-
YFLFLFNLIFFILGAVILGFGV
SEQ ID NO: 37-
MGAYVFIGVGAVTMLMGFL
SEQ ID NO: 38-
LLGLYFAFLLLILIAQVTAGALFYFNM
SEQ ID NO: 39-
GIILGVGVGVAIIELLGMVLSI

The transmembrane domain may be a variant of a transmembrane domain from CD82. The variant may have at least 80, 85, 90, 95, 98 or 99% sequence identity to SEQ ID NO: 36, 37, 38 or 39, provided that the variant sequence retains the capacity to traverse the membrane.

The multi-span CAR may comprise transmembrane domains from different proteins. For example, the multi-span CAR may comprise at least one transmembrane domain derived from CD20, CD53, CD81, CD82 and/or CD28. For example, the multi-span CAR may comprise at least one transmembrane domain derived from CD20 and a transmembrane domain derived from CD28. For example, the multi-span CAR may comprise four transmembrane domains derived from CD20, CD53, CD81 or CD82 and a transmembrane domain derived from CD28. For example, the multi-span CAR may comprise four transmembrane domains derived from CD20 and a transmembrane domain derived from CD28. For example, the multi-span CAR may comprise four transmembrane domains derived from CD53 and a transmembrane domain derived from CD28. For example, the multi-span CAR may comprise four transmembrane domains derived from CD81 and a transmembrane domain derived from CD28. For example, the multi-span CAR may comprise four transmembrane domains derived from CD82 and a transmembrane domain derived from CD28.

Suitably, the multi-span CAR may comprise the sequence shown as positions 271 to 759 or 346 to 759 of SEQ ID NO: 1, 2 or 3, or position 270 to the terminus of SEQ ID NO: 40 to 45 or position 362 to the terminus of SEQ ID NO: 40 to 45 or a variant which shares at least 80% sequence identity thereto.

In one embodiment, the multi-span CAR comprises the sequence shown as SEQ ID NO: 1, 2, 3, or 41 to 45 or a variant which shares at least 80% sequence identity thereto.

In one embodiment, the multi-span CAR comprises the sequence shown as positions 346 to 759 of SEQ ID NO: 1, 2 or 3 or a variant which shares at least 80% sequence identity with the sequence shown as positions 346 to 759 of SEQ ID NO: 1, 2 or 3. In one embodiment, the multi-span CAR comprises the sequence shown as positions 271 to 759 of SEQ ID NO: 1, 2 or 3 or a variant which shares at least 80% sequence identity with the sequence shown as positions 271 to 759 of SEQ ID NO: 1, 2 or 3. The multi-span CAR may comprise the sequence shown as SEQ ID NO: 1, 2 or 3 or a variant which shares at least 80% sequence identity with SEQ ID NO: 1, 2 or 3.

In one embodiment, the multi-span CAR comprises the sequence shown as positions 270 to 698 or 346 to 698 of SEQ ID NO: 40 or positions 270 to 677 or 346 to 677 of SEQ ID NO: 41; or a variant which shares at least 80% sequence identity with the sequence thereto. The multi-span CAR may comprise the sequence shown as SEQ ID NO: 40 or 41 or a variant which shares at least 80% sequence identity with SEQ ID NO: 40 or 41.

In one embodiment, the multi-span CAR comprises the sequence shown as positions 270 to 715, or 346 to 715 of SEQ ID NO: 42 or positions 270 to 699, or 346 to 699 of SEQ ID NO: 43; or a variant which shares at least 80% sequence identity with the sequence thereto. The multi-span CAR may comprise the sequence shown as SEQ ID NO: 42 or 43 or a variant which shares at least 80% sequence identity with SEQ ID NO: 42 or 43.

In one embodiment, the multi-span CAR comprises the sequence shown as positions 270 to 708, or 346 to 708 of SEQ ID NO: 44 or positions 270 to 725, or 346 to 725 of SEQ ID NO: 45; or a variant which shares at least 80% sequence identity with the sequence thereto. The multi-span CAR may comprise the sequence shown as SEQ ID NO: 44 or 45 or a variant which shares at least 80% sequence identity with SEQ ID NO: 44 or 45.

The above-mentioned variants may share at least 80, at least 85, at least 90, at least 95, at least 98 or at least 99% sequence identity to the sequence to which they are compared above.

Multiple algorithms are available which allow the determination of whether a residue in a multi-span transmembrane protein is extracellular, intracellular or transmembrane. An illustrative list of such algorithms includes TMHMM (Krogh et al.; 2001; J. Mol. Biol; 305; 567-580); OCTOPUS (Viklund, H. & Elofsson;. 2008; Bioinformatics 24: 1662-1668); and Phobius (L. Käll et al.; 2004; J. Mol. Biol. 338: 1027-1036). Thus, methods for analysing a multi-span transmembrane protein and determining protein topology (i.e. which residues are intracellular, extracellular and transmembrane) are well known in the art.

The transmembrane domains of the multi-span CAR may be linked by any suitable amino acid sequence. For example, the transmembrane domains may be linked by the extracellular or intracellular loops of a native multi-span transmembrane protein.

A multi-span CAR of the present invention may comprise the transmembrane domains and the intra- and extra-cellular loops of a native multi-span transmembrane protein. The multi-span CAR of the present invention may lack functional domains compared to a corresponding native multi-span transmembrane protein.

By way of example, if a multi-span CAR has an antigen binding domain located at the amino- or carboxyterminus of a native multi-span transmembrane protein, the amino- or carboxy-terminal transmembrane domain of the native multi-span transmembrane protein may be deleted and replaced with the antigen-binding domain and transmembrane domain of a classical CAR structure (e.g. an antigen-binding domain and a transmembrane domain as described herein).

By way of example, if a native multi-span transmembrane protein comprises a native antigen-binding domain (e.g. a ligand binding domain), this native antigen-binding domain may be deleted to produce a multi-span transmembrane protein which is not capable of binding its native ligand in the context of the multi-span CAR. As such, the multi-span CAR is only able to bind antigen via the CAR antigen-binding domain, as described herein. By way of further example, if the native multi-span transmembrane protein comprises a native signalling domain, this native signalling domain may be deleted to produce a multi-span transmembrane protein which is only capable of signalling via the CAR intracellular signalling domain, as described herein, when in the context of a multi-span CAR. By way of even further example, if a native multi-span transmembrane protein comprises a native antigen-binding domain (e.g. a ligand binding domain) and a native signalling domain—each of the native antigen-binding domain and the native signalling domain may be deleted such that only the transmembrane domains and the intra- and extra-cellular loops of the native multi-span transmembrane domain are present in the multi-span CAR.

Antigen Binding Domain

The antigen-binding domain is the portion of a classical CAR which recognizes antigen. In the multi-span CAR of the present invention the antigen-binding domain is located at an extracellular domain of the multi-span CAR. The antigen-binding domain of the multi-span CAR is therefore orientated on the extracellular side of the membrane when expressed in a cell.

As used herein, an extracellular domain of a multi-span CAR may refer to an amino-terminal extracellular domain, a carboxy-terminal extracellular domain or an extracellular loop located between two adjacent transmembrane domains in the primary amino acid sequence.

The antigen binding domain may be located at any extracellular domain of the multi-span CAR.

An extracellular domain of a multi-span transmembrane protein may be identified, for example, by using topology algorithms such as those described herein.

The antigen-binding domain may be located as an N-terminal or C-terminal addition to a multi-span transmembrane protein. When located as an N- or C-terminal addition, the wild-type sequence of a native multi-span transmembrane protein may be truncated such that the wild-type sequence ends as an intracellular domain. Here the CAR antigen-binding domain may be added as a structure which is analogous to a classical CAR protein but lacking an intracellular signalling domain. That is, an antigen-binding domain and a transmembrane domain may be added to the amino or carboxy terminus of the truncated multi-span transmembrane protein.

The antigen-binding domain may be located at an extracellular loop of the multi-span CAR. Here, the antigen-binding domain is located between two adjacent transmembrane domains of the multi-span CAR such that it is located on the extracellular side of the cell membrane when the multi-span CAR is expressed in a cell. The antigen-binding domain may be inserted into the wild-type sequence of an extracellular domain from a native multi-span transmembrane protein. The antigen-binding domain may replace the wild-type sequence of an extracellular domain from a native multi-span transmembrane protein.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Multiple Antigen-Binding Domains

The multi-span CAR of the present invention may comprise more than one antigen-binding domain. Each antigen-binding domain may be located at a different extracellular domain of the multi-span CAR.

Suitably, the multi-span CAR of the present invention may comprise at least two, at least three, or at least four antigen-binding domains. Suitably, the multi-span CAR of the present invention may comprise from two up to four antigen-binding domains.

By way of example, the multi-span CAR may comprise two, three, four or more antigen-binding domains.

The multi-span CAR may have an antigen-binding domain located at each available extracellular domain. The multi-span CAR may have an antigen-binding domain located at the amino and/or carboxy-termini (if located on the extracelluar side) and at each of the extracellular loops of the multi-span transmembrane protein.

The multi-span CAR may have an antigen-binding domain located at a subset of the available extracellular domains. By way of example, the multi-span CAR may have an antigen-binding domain located at the extracellular amino-terminal and a single extracellular loop of the multi-span transmembrane protein.

Each of the multiple antigen-binding domains may recognise different antigens. Such a multi-span CAR would be capable of recognizing multiple antigens. This might be useful for instance in avoiding tumour escape.

Spacer Domain

The multi-span CAR according to the present invention may comprise spacer sequences to connect the antigen-binding domain with adjacent transmembrane domains. A flexible spacer allows the antigen-binding domain to orient in different directions to facilitate binding.

Where the antigen binding domain is located at the N-terminus or C-terminus of the multi-span CAR, a spacer may be included between the antigen binding domain and the transmembrane domain. Where the antigen binding domain is located at an extracellular loop, the antigen binding domain may be connected to the transmembrane domain on the amino side by a first spacer and to the transmembrane domain on the carboxy side by a second spacer. Where more than one spacer is used, the spacer sequences may be the same or different.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Examples of amino acid sequences for these spacers are given below:

SEQ ID NO: 5 (hinge-CH2CH3 of human IgG1)
AEPKSPDKTHTCPPCPAPPVAGPSVFLFPPKPKDTLMIARTPEVTCVV
VDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELT
KNQVSLTCLVKGFYPSDIAVEWESNGQPENNYKTTPPVLDSDGSFFLY
SKLTVDKSRWQQGNVFSCSVMHEALHNHYTQKSLSLSPGKKD
SEQ ID NO: 6 (human CD8 stalk):
TTTPAPRPPTPAPTIASQPLSLRPEACRPAAGGAVHTRGLDFACDI
SEQ ID NO: 7 (human IgG1 hinge):
AEPKSPDKTHTCPPCPKDPK
SEQ ID NO: 8 (CD2 ectodomain)
KEITNALETWGALGQDINLDIPSFQMSDDIDDIKWEKTSDKKKIAQFR
KEKETFKEKDTYKLFKNGTLKIKHLKTDDQDIYKVSIYDTKGKNVLEK
IFDLKIQERVSKPKISWTCINTTLTCEVMNGTDPELNLYQDGKHLKLS
QRVITHKWTTSLSAKFKCTAGNKVSKESSVEPVSCPEKGLD
SEQ ID NO: 9 (CD34 ectodomain)
SLDNNGTATPELPTQGTFSNVSTNVSYQETTTPSTLGSTSLHPVSQHG
NEATTNITETTVKFTSTSVITSVYGNTNSSVQSQTSVISTVFTTPANV
STPETTLKPSLSPGNVSDLSTTSTSLATSPTKPYTSSSPILSDIKAEI
KCSGIREVKLTQGICLEQNKTSSCAEFKKDRGEGLARVLCGEEQADAD
AGAQVCSLLLAQSEVRPQCLLLVLANRTEISSKLQLMKKHQSDLKKLG
ILDFTEQDVASHQSYSQKT
INTRACELLULAR SIGNALLING DOMAIN

The intracellular signalling domain is the signal-transmission portion of a classical CAR. In the multi-span CAR of the present invention, the intracellular signalling domain is located at an intracellular domain of the multi-span CAR. As such, each intracellular signalling domain of the multi-span CAR of the present invention is equivalent to the single intracellular domain of a classical CAR based on a single transmembrane domain protein.

The intracellular signalling domain may be located at any intracellular domain of the multi-span CAR.

An intracellular domain of a multi-span transmembrane protein may be identified, for example, by using topology algorithms such as those described herein.

The intracellular signalling domain may be located at the N-terminal or C-terminal of the multi-span CAR, if located on the intracellular side. For example, the intracellular signalling domain may be located as an N-terminal or C-terminal extension to a native multi-span transmembrane protein. When the intracellular signalling domain is located as an N- or C-terminal extension, the wild-type sequence of a native multi-span transmembrane protein may be truncated.

The intracellular signalling domain may be located at an intracellular loop of the multi-span CAR. Here, the intracellular signalling domain is located between two adjacent transmembrane domains of the multi-span CAR and is orientated on the intracellular side of the membrane when the multi-span CAR is expressed in a cell. The intracellular signalling domain may be inserted into the sequence of the intracellular domain of a native multi-span transmembrane protein. The intracellular signalling domain may replace the native sequence of the intracellular domain of a native multi-span transmembrane protein.

The intracellular signalling domain may comprise a single signalling endodomain.

The most commonly used signalling domain component is that of CD3-zeta endodomain, which contains 3 immunoreceptor tyrosine-based activation motifs (ITAMs). This transmits an activation signal to a T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in the context of a classical CAR in FIG. 1B).

The intracellular signalling domain of the multi-span CAR of the present invention may comprise a single endodomain selected from CD3 zeta endodomain, CD28 endodomain, 41BB endodomain, an OX40 endodomain, CD2 endodomain, Inducible T-cell costimulator (ICOS) endodomain, CD27 endodomain, BTLA endodomain, CD30 endodomain, GITR endodomain or HVEM endodomain.

The intracellular signalling domain of the multi-span CAR of the present invention may comprise a single endodomain selected from CD3 zeta endodomain, CD28 endodomain, 41BB endodomain and an OX40 endodomain

The intracellular signalling domain may comprise the CD3-Zeta endodomain alone

The endodomain signalling component may comprise the sequence shown as SEQ ID NO: 10 to 13 or a variant thereof having at least 80% sequence identity.

SEQ ID NO: 10- CD3 Z endodomain
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR
SEQ ID No. 11- CD28 endodomain
KRSRLLHSDYMNMTPRRPGPTRKHYQPYAPPRDFAAY
SEQ ID No. 12- OX40 endodomain
RRDQRLPPDAHKPPGGGSFRTPIQEEQADAHSTLAKI
SEQ ID No. 13- 41BB endodomain
KRGRKKLLYIFKQPFMRPVQTTQEEDGCSCRFPEEEEGGCEL

The intracellular signalling domain may comprise a plurality of constituent signalling endodomains. By way of example, each intracellular signalling domain may comprise two, three or four or more signalling endodomains. The combination of multiple signalling domains is also referred to herein as a compound signalling domain.

The intracellular signalling domain may comprise the CD3-Zeta endodomain together with any one of CD28, 41BB or OX40. The intracellular signalling domain may comprise the CD3-Zeta endodomain, the CD28 endodomain and the 41BB domain. The intracellular signalling domain may comprise the CD3-Zeta endodomain, the CD28 endodomain and the OX40 endodomain.

The intracellular signalling domain may comprise the sequence shown as SEQ ID NO: 14 to 16 or SEQ ID NO: 48 or a variant thereof having at least 80% sequence identity.

SEQ ID NO: 14- CD28 and CD3 Zeta endodomains
SKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQE
GLYNELQKDKMAEAYSEIGMKGERRRGKGHDGLYQGLSTATKDTYDAL
HMQALPPR
SEQ ID NO: 15- CD28, OX40 and CD3 Zeta endodomains
SKRSRLLHSDYMNMTPRRPGPTRKHYQPYAPPRDFAAYRSRDQRLPPD
AHKPPGGGSFRTPIQEEQADAHSTLAKIRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID NO: 16- 41BB, OX40 and CD3 Zeta endodomains
KRGRKKLLYIFKQPFMRPVQTTQEEDGCSCRFPEEEEGGCELRRDQRL
PPDAHKPPGGGSFRTPIQEEQADAHSTLAKIRVKFSRSADAPAYQQGQ
NQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 48- 41BB CD3 Zeta endodomains
KRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNP
QEGLYNELQKDKMAEAYSEIGMKGERRRGKGHDGLYQGLSTATKDTYD
ALHMQALPPR

A variant sequence may have at least 80%, 85%, 90%, 95%, 98% or 99% sequence identity to SEQ ID NO: 10 to 16 or SEQ ID NO: 48 provided that the sequence provides an effective intracellular signalling domain.

Multiple Intracellular Signalling Domains

The multi-span CAR of the present invention may comprise more than one intracellular signalling domains. In other words, the multi-span CAR may comprise intracellular signalling domains located at different intracellular domains of the multi-span CAR.

By way of example, the multi-span CAR may comprise at least two, at least three or at least four intracellular signalling domains.

The multi-span CAR may comprise two, three, four, five or more intracellular signalling domains.

The multi-span CAR may have an intracellular signalling domain located at each available intracellular domain of the multi-span transmembrane protein. The multi-span CAR may have an intracellular signalling domain located at the amino and/or carboxy-termini (if located on the intracellular side) and at each of the intracellular loops.

The multi-span CAR may have an intracellular signalling domain located at a subset of the available intracellular domains. By way of example, the multi-span CAR may have an intracellular signalling domain located at the intracellular carboxy-terminal and a single intracellular loop. By way of further example, the multi-span CAR may have an intracellular signalling domain located at the intracellular carboxy-terminal and more than one, for example at two or three, intracellular loops.

The multiple intracellular signalling domains may each comprise the same constituent signalling endodomain(s). Alternatively, a subset of the multiple intracellular signalling domains may comprise the same constituent signalling endodomain(s) whilst other intracellular signalling domains comprise different constituent signalling endodomain(s). Alternatively, the multiple intracellular signalling domains may each comprise different constituent signalling endodomain(s).

In one embodiment, the multi-span CAR may comprise a 41BB endodomain at two different intracellular loops and a CD3-Zeta endodomain at the intracellular carboxy-terminal. In one embodiment, the multi-span CAR may comprise a 41BB endodomain at one intracellular loop, a CD28 endodomain at a different intracellular loop and a CD3-Zeta endodomain at the intracellular carboxy-terminal. The CD3-Zeta endomain may be a 41BB/CD3-Zeta compound endodomain (e.g. as shown in SEQ ID NO: 48, or a variant of SEQ ID NO: 48 sharing at least 80, 85, 90, 95, 98 or 99% sequence identity with SEQ ID NO: 48).

Each of the multiple intracellular signalling domains may comprise the CD3-Zeta endodomain, the CD28 endodomain and the 41BB domain. Each of the multiple intracellular signalling domains may comprise the CD3-Zeta endodomain, the CD28 endodomain and the OX40 endodomain.

Suitably, the multi-span CAR may comprises the sequence shown as positions 270 to 789, or 346 to 708 of SEQ ID NO: 46 or positions 270 to 788, or 346 to 788 of SEQ ID NO: 47; or a variant which shares at least 80% sequence identity with the sequence thereto. The multi-span CAR may comprise the sequence shown as SEQ ID NO: 46 or 47 or a variant which shares at least 80% sequence identity with SEQ ID NO: 46 or 47.

At least 80% sequence identity means that the variant may share at least 80, at least 85, at least 90, at least 95, at least 98 or at least 99% sequence identity with the sequence to which it is compared above.

Signal Peptide

The multi-span CAR of the present invention may comprise a signal peptide so that when the CAR is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the sequence shown as SEQ ID NO: 17 to 19 or a variant thereof having 5, 4, 3, 2 or 1 amino acid mutations (insertions, substitutions or additions) provided that the signal peptide still functions to cause cell surface expression of the CAR.

SEQ ID NO: 17:
MGTSLLCWMALCLLGADHADG

The signal peptide of SEQ ID NO: 17 is compact and highly efficient. It is predicted to give about 95% cleavage after the terminal glycine, giving efficient removal by signal peptidase.

SEQ ID NO: 18:
MSLPVTALLLPLALLLHAARP

The signal peptide of SEQ ID NO: 18 is derived from IgG1.

SEQ ID NO: 19:
MAVPTQVLGLLLLWLTDARC

The signal peptide of SEQ ID NO: 19 is derived from CD8.

Nucleic Acid

The present invention further provides a nucleic acid encoding a multi-span CAR according to the present invention.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid of the invention may be a nucleic acid which encodes a multi-span CAR according to the present invention and at least a second polypeptide. By way of example, the second polypeptide may be a marker/suicide polypeptide as described herein.

Where the nucleic acid encodes a multi-span CAR according to the present invention and at least a second polypeptide the nucleic acid may encode a polypeptide which comprises the multi-span CAR and the second polypeptide joined by a cleavage site. The cleavage site may be self-cleaving, such that when the polypeptide is produced, it is immediately cleaved into the multi-span CAR and the second polypeptide without the need for any external cleavage activity.

For example, the present invention provides a nucleic acid sequence encoding a multi-span CAR of the invention, wherein the nucleic acid sequence comprises the following structure:

A-X—B

in which A is the nucleic acid sequence encoding a multi-span CAR of the invention, B is a nucleic acid sequence encoding a second polypeptide; and X is a nucleic acid sequence which encodes a cleavage site, such that A is cleaved from B after translation.

The cleavage site may be any sequence which enables the polypeptides encoded by each of A and B to become separated.

The term “cleavage” is used herein for convenience, but the cleavage site may cause the targeting component and the signalling component to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode targeting component and the signalling component, causes the targeting component and the signalling component to be expressed as separate entities.

The cleavage site may be a furin cleavage site or a Tobacco Etch Virus (TEV) cleavage site. The cleavage site may encode a self-cleaving peptide.

A ‘self-cleaving peptide’ refers to a peptide which functions such that when the polypeptide comprising the targeting component and the signalling component and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FM DV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences.

The cleavage site may comprise the 2A-like sequence shown as SEQ ID NO: 20 (RAEGRGSLLTCGDVEENPGP).

Vector

The present invention also provides a vector which comprises a nucleic acid sequence encoding a multi-span CAR of the present invention. Such a vector may be used to introduce the nucleic acid sequence into a host cell so that it expresses the multi-span CAR of the present invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention also relates to a cell, such as an immune cell comprising the multi-span CAR of the present invention.

The cell may be a T cell, NK cell, γδ T cell, myeloid cell or macrophage. In a preferred embodiment the cell is a T cell or NK cell.

The cell may comprise a nucleic acid or a vector of the present invention.

The cell may be a T cell. T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

T or NK cells expressing a multi-span CAR according to the present invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells expressing a multi-span CAR according to the present invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CAR cells are generated by introducing DNA or RNA coding for the targeting component and signalling component by one of many means including transduction with a viral vector, transfection with DNA or RNA.

The CAR cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the CAR according to the present invention, for example by treatment with an anti-CD3 monoclonal antibody.

A cell of the invention, for example a T cell or a NK cell may be made by:
(i) isolation of a cell-containing sample from a subject or other sources listed above; and
(ii) transduction or transfection of the cell with a nucleic acid encoding a multi-span CAR according to the present invention.

The cells may then by purified, for example, selected on the basis of expression of the antigen-binding domain of the antigen-binding polypeptide.

Marker/Suicide Protein

Since immune cells, such as T-cells, engraft and are autonomous, a means of selectively deleting CAR cells in recipients of CAR cells is desirable. Suicide genes are genetically encodable mechanisms which result in selective destruction of infused cells in the face of unacceptable toxicity. The earliest clinical experience with suicide genes is with the Herpes Virus Thymidine Kinase (HSV-TK) which renders cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. The biggest limitation of iCasp9 is dependence on availability of clinical grade proprietary CID. Both iCasp9 and HSV-TK are intracellular proteins, so when used as the sole transgene, they have been co-expressed with a marker gene to allow selection of transduced cells.

An iCasp9 may comprise the sequence shown as SEQ ID NO: 21 or a variant thereof having at least 80, 90, 95 or 98% sequence identity.

SEQ ID NO: 21
MLEGVQVETISPGDGRTFPKRGQTCVVHYTGMLEDGKKVDSSRDRNKP
FKFMLGKQEVIRGWEEGVAQMSVGQRAKLTISPDYAYGATGHPGIIPP
HATLVFDVELLKLESGGGSGVDGFGDVGALESLRGNADLAYILSMEPC
GHCLIINNVNFCRESGLRTRTGSNIDCEKLRRRFSSLHFMVEVKGDLT
AKKMVLALLELAQQDHGALDCCVVVILSHGCQASHLQFPGAVYGTDGC
PVSVEKIVNIFNGTSCPSLGGKPKLFFIQACGGEQKDHGFEVASTSPE
DESPGSNPEPDATPFQEGLRTFDQLDAISSLPTPSDIFVSYSTFPGFV
SWRDPKSGSWYVETLDDIFEQWAHSEDLQSLLLRVANAVSVKGIYKQM
PGCFNFLRKKLFFKTSAS

A recently described novel marker/suicide gene is RQR8, which can be detected with the antibody QBEnd10 and cells expressing RQR8 can be selectively lysed with the therapeutic antibody Rituximab.

An RQR8 may comprise the sequence shown as SEQ ID NO: 22 or a variant thereof having at least 80, 90, 95 or 98% sequence identity.

SEQ ID NO: 22
MGTSLLCWMALCLLGADHADACPYSNPSLCSGGGGSELPTQGTFSNVS
TNVSPAKPTTTACPYSNPSLCSGGGGSPAPRPPTPAPTIASQPLSLRP
EACRPAAGGAVHTRGLDFACDIYIWAPLAGTCGVLLLSLVITLYCNHR
NRRRVCKCPRPVV

The suicide gene may be expressed as a single polypeptide with the CAR, for example by using a self-cleaving peptide between the two sequences.

Pharmaceutical Composition

The present invention also provides a pharmaceutical composition containing a cell according to the present invention and a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The cell-containing sample may be isolated from a subject or from other sources, for example as described above. The cell may be isolated from a subject's own peripheral blood (1st party), or in the setting of a haematopoietic stem cell transplant from donor peripheral blood (2nd party), or peripheral blood from an unconnected donor (3rd party).

The present invention provides a multi-span CAR of the present invention for use in treating and/or preventing a disease. For example the present invention provides nucleic acid or a vector encoding a multi-span CAR according to the present invention or a cell comprising a multi-span CAR according to the present invention for use in treating and/or preventing a disease.

The invention also relates to the use of a multi-span CAR of the present invention in the manufacture of a medicament for the treatment and/or prevention of a disease. For example the present invention provides the use of a nucleic acid or a vector encoding a multi-span CAR according to the present invention or a cell comprising a multi-span CAR according to the present invention in the manufacture of a medicament for the treatment and/or prevention of a disease.

The disease to be treated and/or prevented by the methods of the present invention may be an infection, such as a viral infection.

The methods of the invention may also be for the control of pathogenic immune responses, for example in autoimmune diseases, allergies and graft-vs-host rejection.

The methods may be for the treatment of a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The CAR cells of the present invention may be capable of killing target cells, such as cancer cells. The target cell may be recognisable by expression of a TAA, for example the expression of a TAA provided above in Table 1.

The CAR cells and pharmaceutical compositions of present invention may be for use in the treatment and/or prevention of the diseases described above.

The CAR cells and pharmaceutical compositions of present invention may be for use in any of the methods described above.

The term “nucleotide sequence” as used herein refers to an oligonucleotide sequence or polynucleotide sequence, and variant, homologues, fragments and derivatives thereof (such as portions thereof). The nucleotide sequence may be synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232-incorporated herein by reference).

The present nucleotide sequence may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805-incorporated herein by reference. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491)—incorporated herein by reference.

The scope of the present invention also encompasses amino acid sequences of enzymes having the specific properties as defined herein.

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homology” can be equated with “identity”.

The homologous amino acid sequence and/or nucleotide sequence and/or fragments should provide and/or encode a polypeptide which retains the functional activity.

Typically, the homologous sequences will comprise the same functoinal sites etc. as the subject amino acid sequence for instance or will encode the same active sites. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In one embodiment the present invention relates to a protein whose amino acid sequence is represented herein or a protein derived from this protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In one embodiment the present invention relates to a nucleic acid sequence (or gene) encoding a protein whose amino acid sequence is represented herein or encoding a protein derived from this protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Homology or identity comparisons can be conducted by eye, or more usually, with the aid of readily available sequence comparison programs. These commercially available computer programs can calculate % homology between two or more sequences.

% homology or % identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.qov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244, incorporated herein by reference).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 0 |
| GAP EXTENSION | 0 |

| FOR CLUSTAL | DNA | PROTEIN | |
|---|---|---|---|
| WORD SIZE | 2 | 1 | K triple |
| GAP PENALTY | 15 | 10 | |
| GAP EXTENSION | 6.66 | 0.1 | |

In one embodiment, CLUSTAL may be used with the gap penalty and gap extension set as defined above.

In some embodiments the gap penalties used for BLAST or CLUSTAL alignment may be different to those detailed above. The skilled person will appreciate that the standard parameters for performing BLAST and CLUSTAL alignments may change periodically and will be able to select appropriate parameters based on the standard parameters detailed for BLAST or CLUSTAL alignment algorithms at the time.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence may be determined over the whole sequence.

As used herein, “at least 80% sequence identity” means that the variant may share at least 80, at least 85, at least 90, at least 95, at least 98 or at least 99% sequence identity with the sequence to which it is compared herein.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone¹⁹⁰ *, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134—incorporated herein by reference

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The term “expression vector” means a construct capable of in vivo or in vitro expression.

Preferably, the expression vector is incorporated into the genome of a suitable host organism. The term “incorporated” preferably covers stable incorporation into the genome.

The nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described herein to provide for expression of a polypeptide of the present invention.

The choice of vector e.g. a plasmid, cosmid, or phage vector will often depend on the host cell into which it is to be introduced.

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions.

Preferably, the nucleotide sequence according to the present invention is operably linked to at least a promoter.

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may contain or express a marker, which allows for the selection of the genetic construct.

Amino acids are referred to herein using the name of the amino acid, the three letter abbreviation or the single letter abbreviation.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents and equivalents thereof known to those skilled in the art, and so forth.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The invention will now be further described by way of Examples, which are meant to serve to assist one of ordinary skill in the art in carrying out the invention and are not intended in any way to limit the scope of the invention.

### EXAMPLES

### Example 1

### Proof of Principle Using a CD20-Based Multi-Span CAR

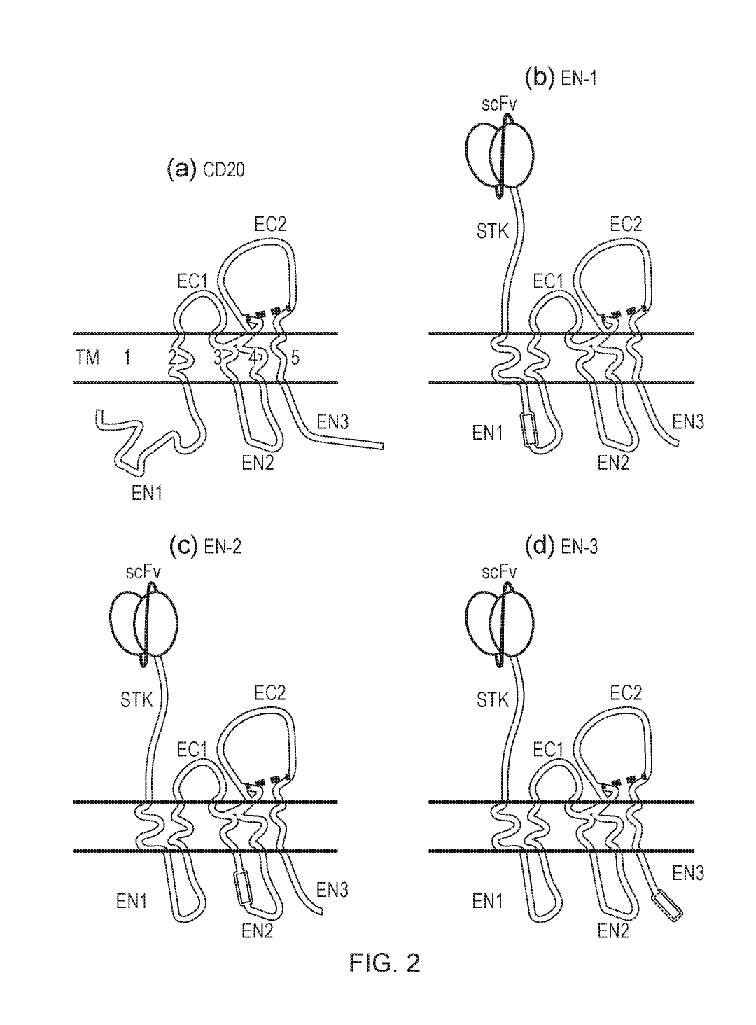

CD20 was analyzed using TMHMM (Krogh et al.; 2001; J. Mol. Biol; 305; 567-580) and used as a scaffold for engineering different multi-span CARs (see FIG. 2).

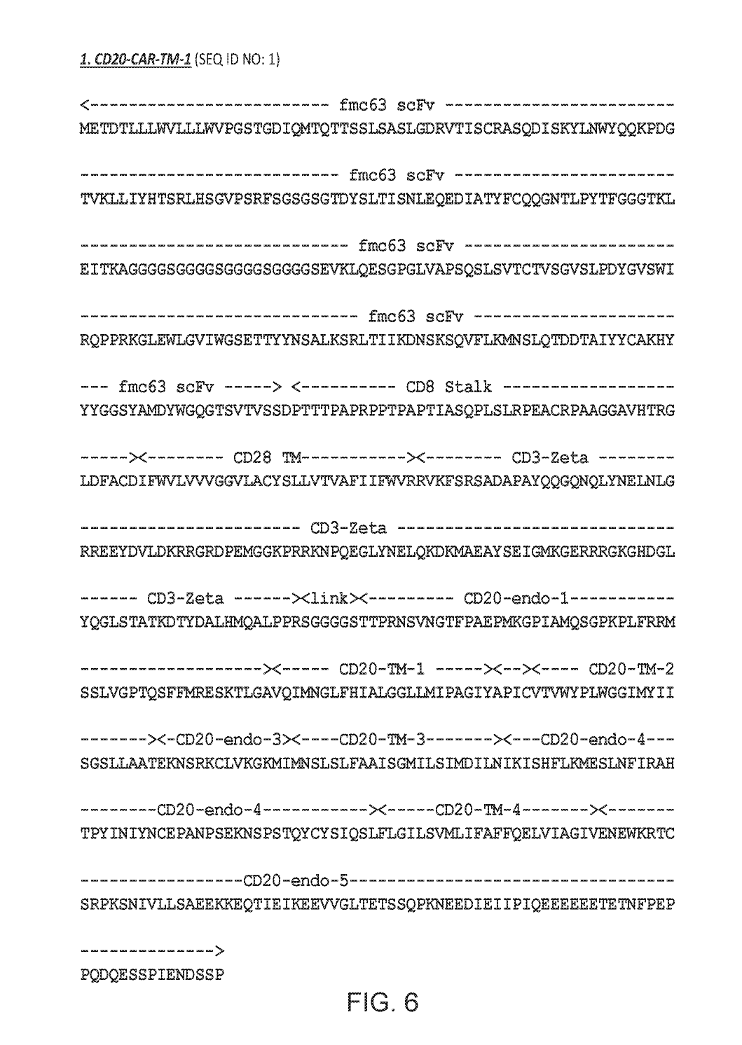

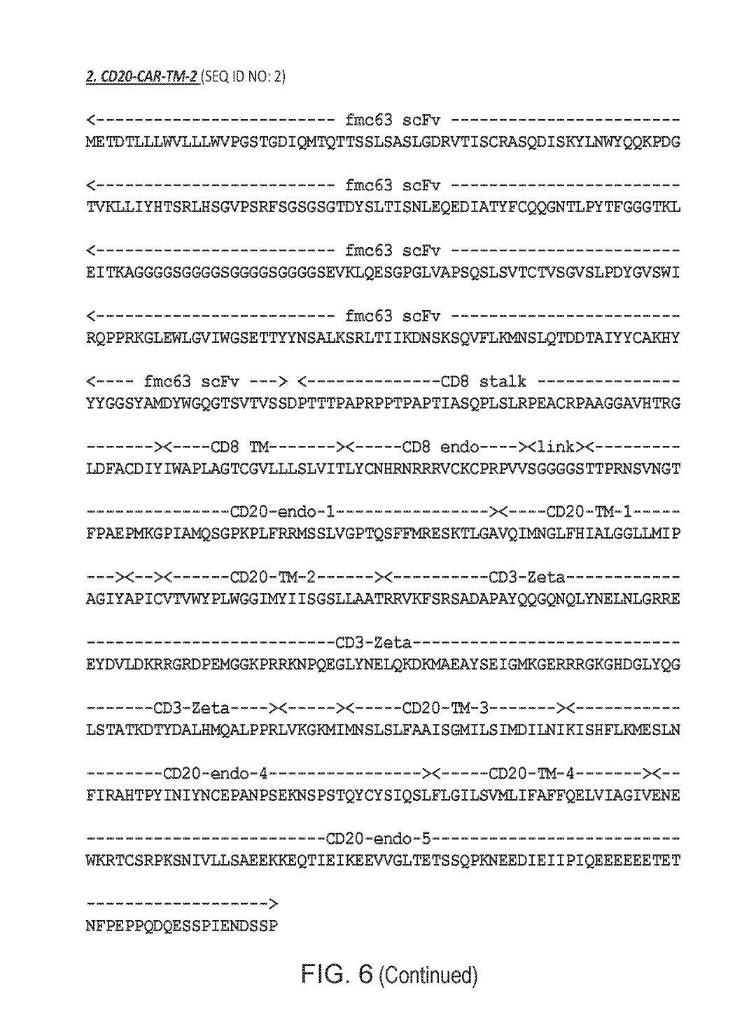

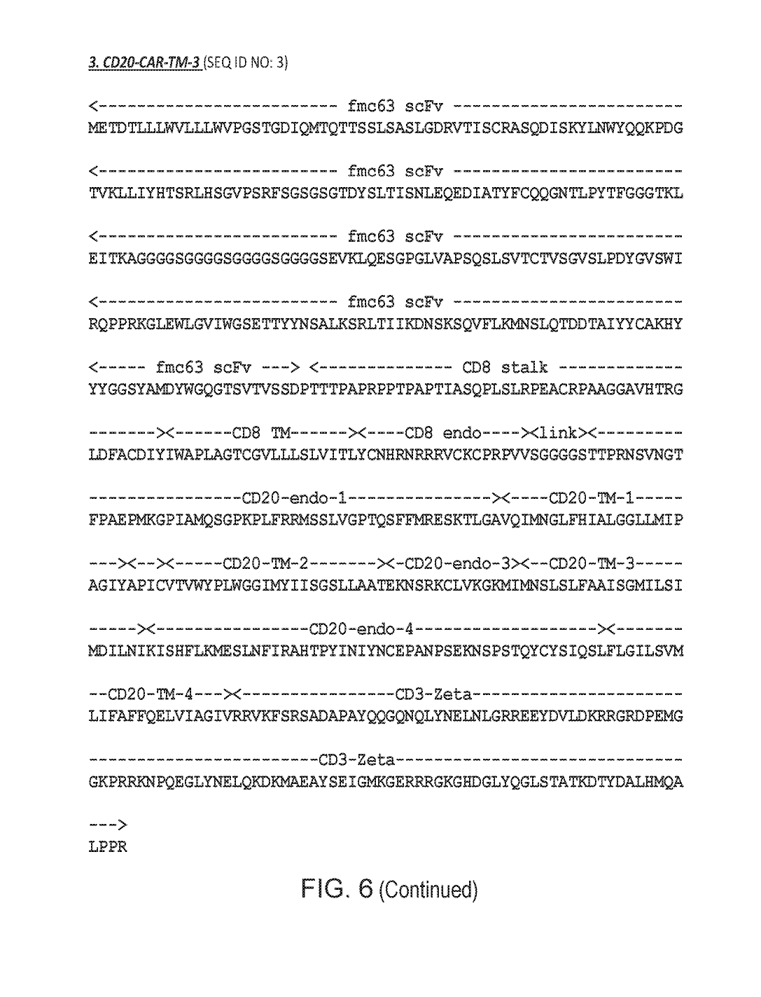

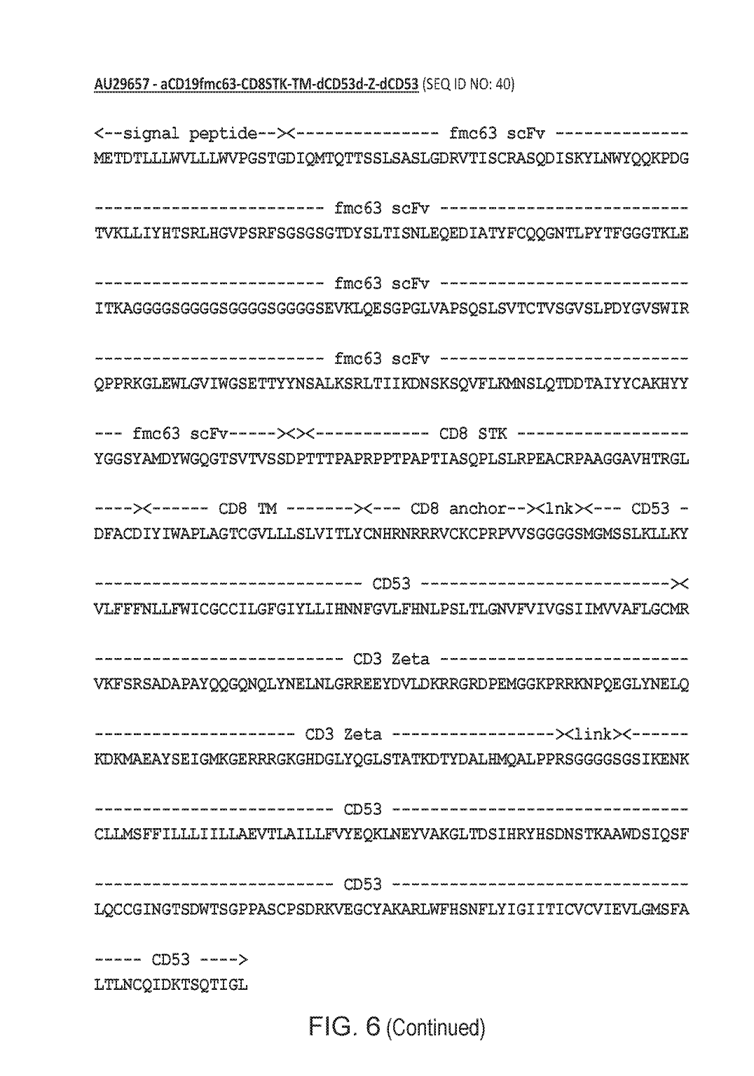

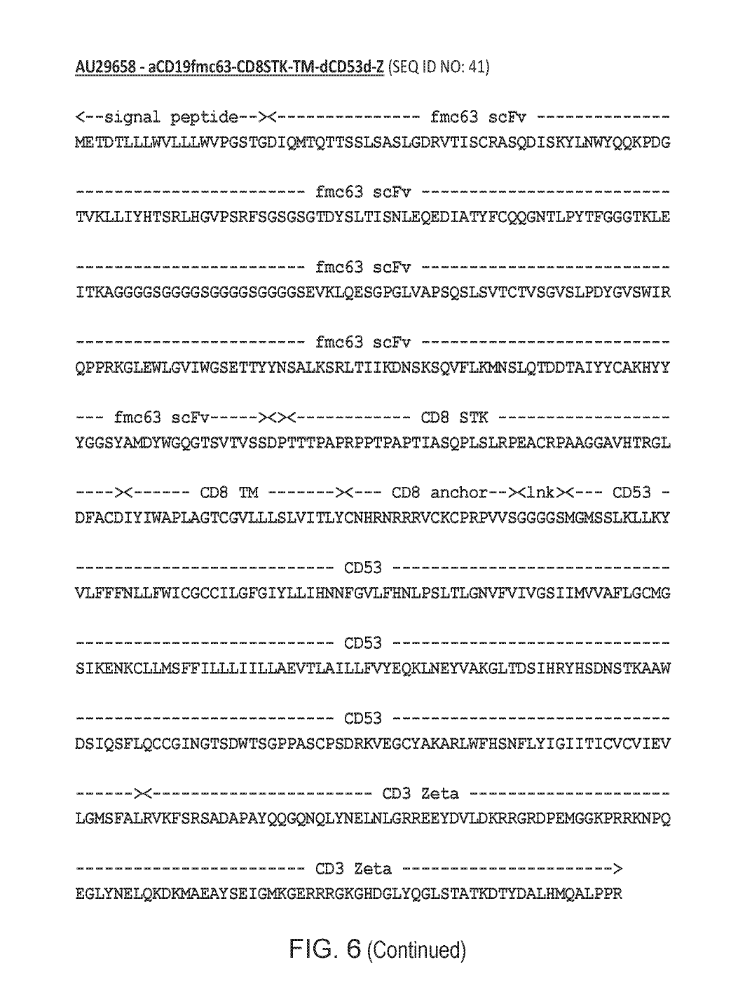

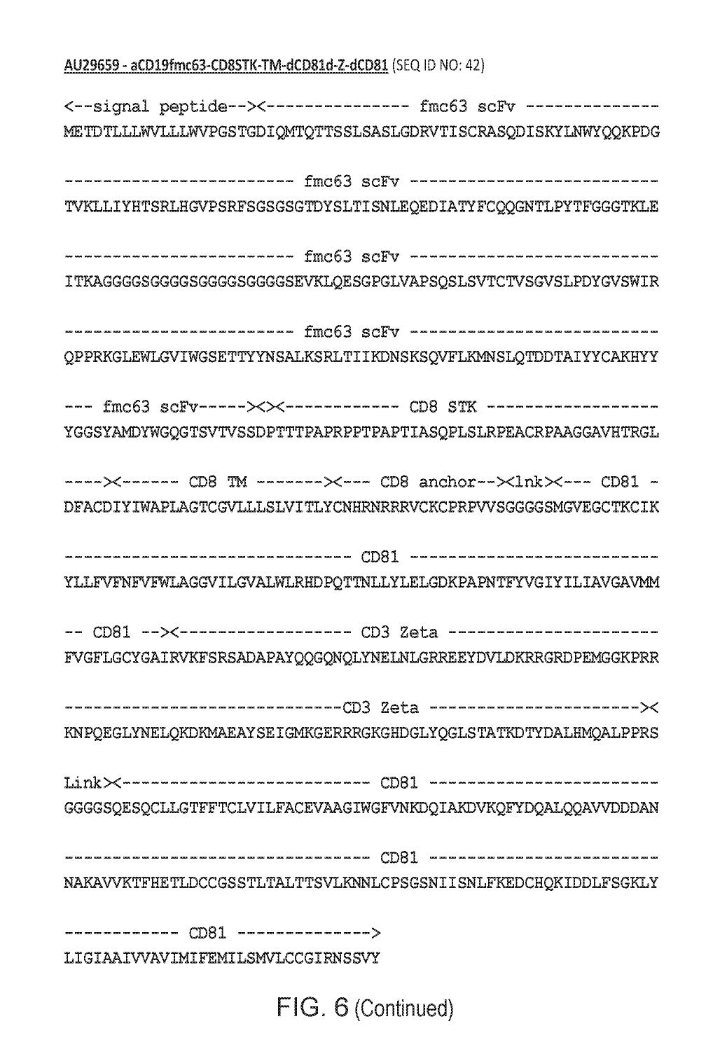

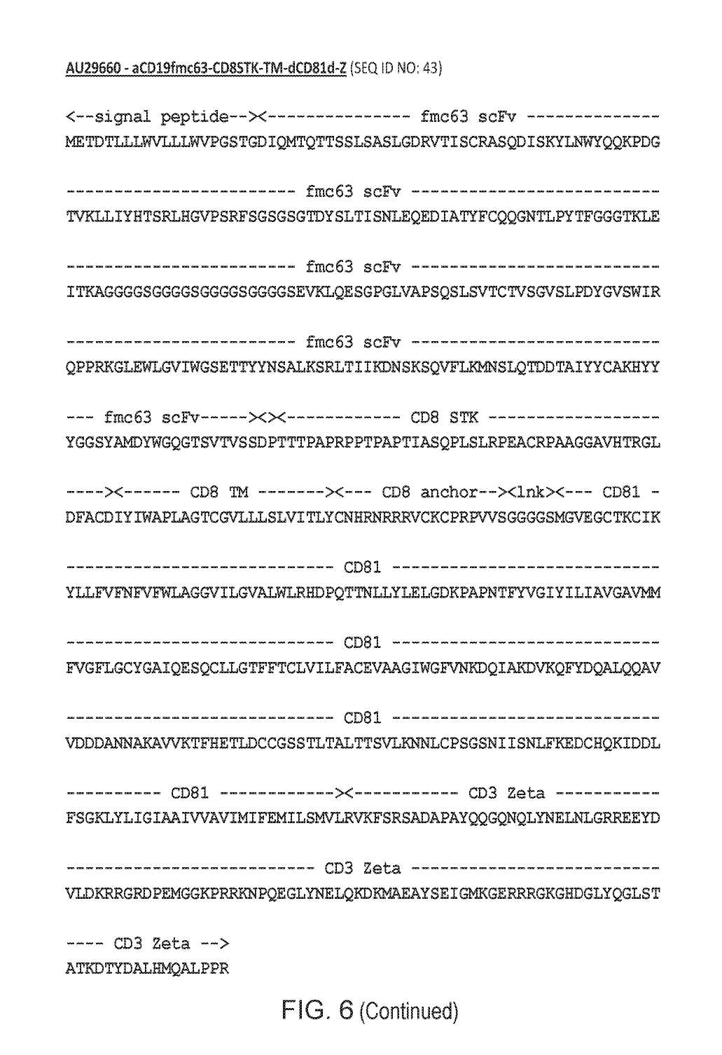

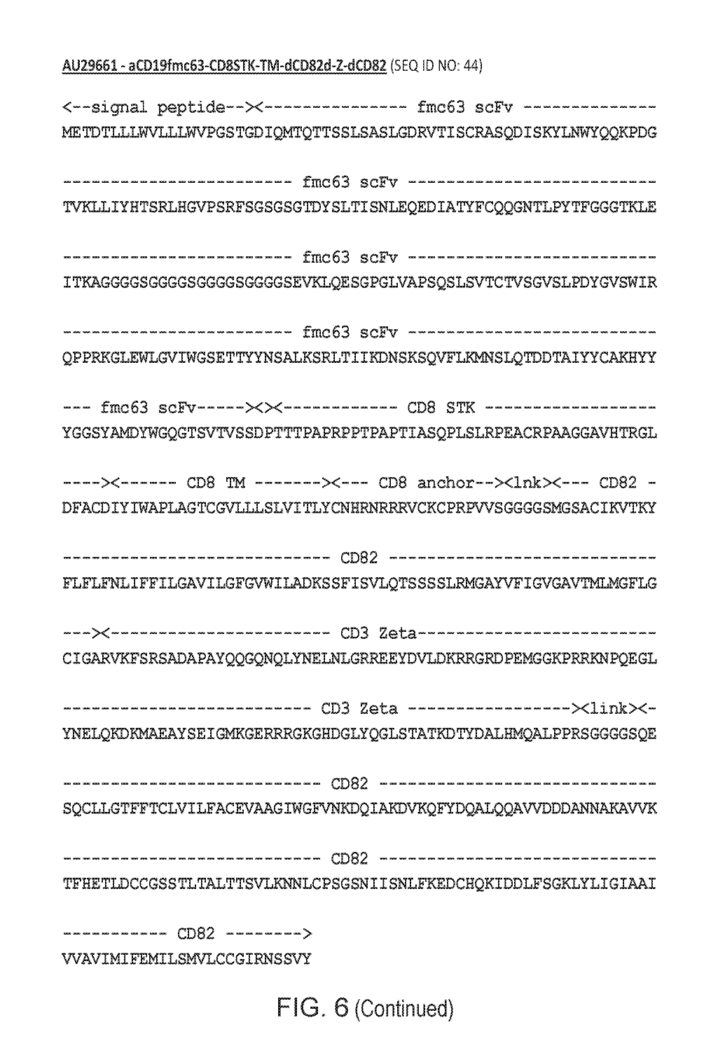

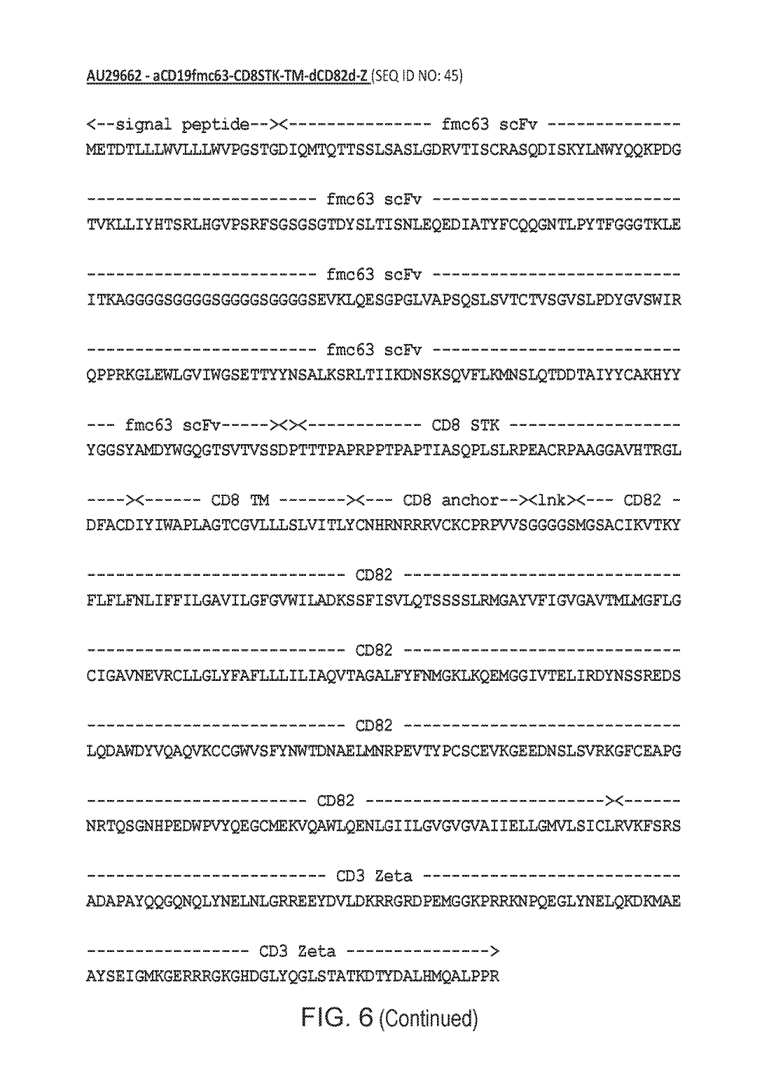

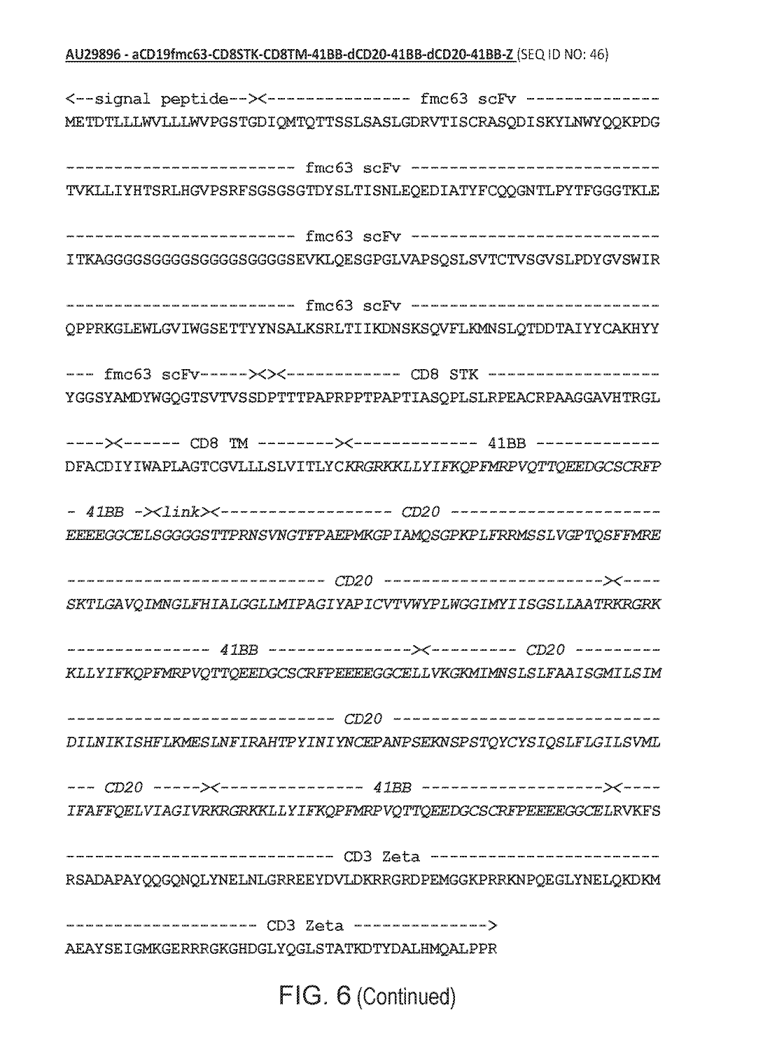

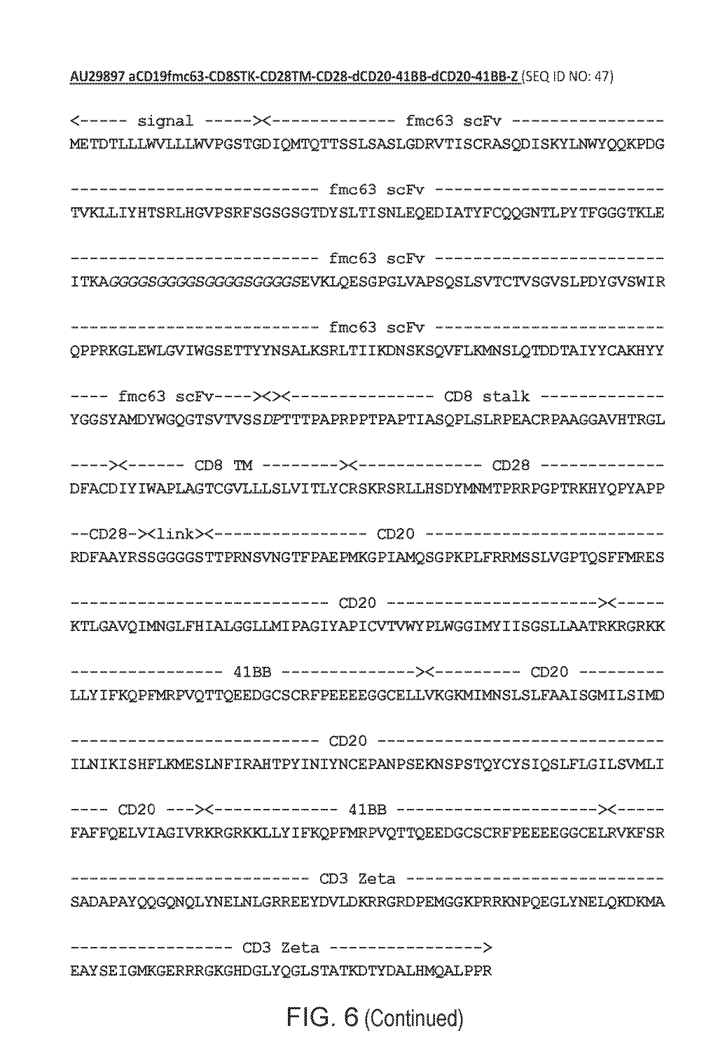

Each of the multi-span CARs illustrated in FIG. 2 had the anti-CD19 scFv fmc63 connected to a CD8 stalk, transmembrane domain and polar anchor to a truncated amino-terminus of CD20. Three variants were constructed with CD3-Zeta in either endodomain 1 (EN1), endodomain 2 (EN2) or endodomain 3 (EN3) (amino acid sequences shown in FIG. 6).

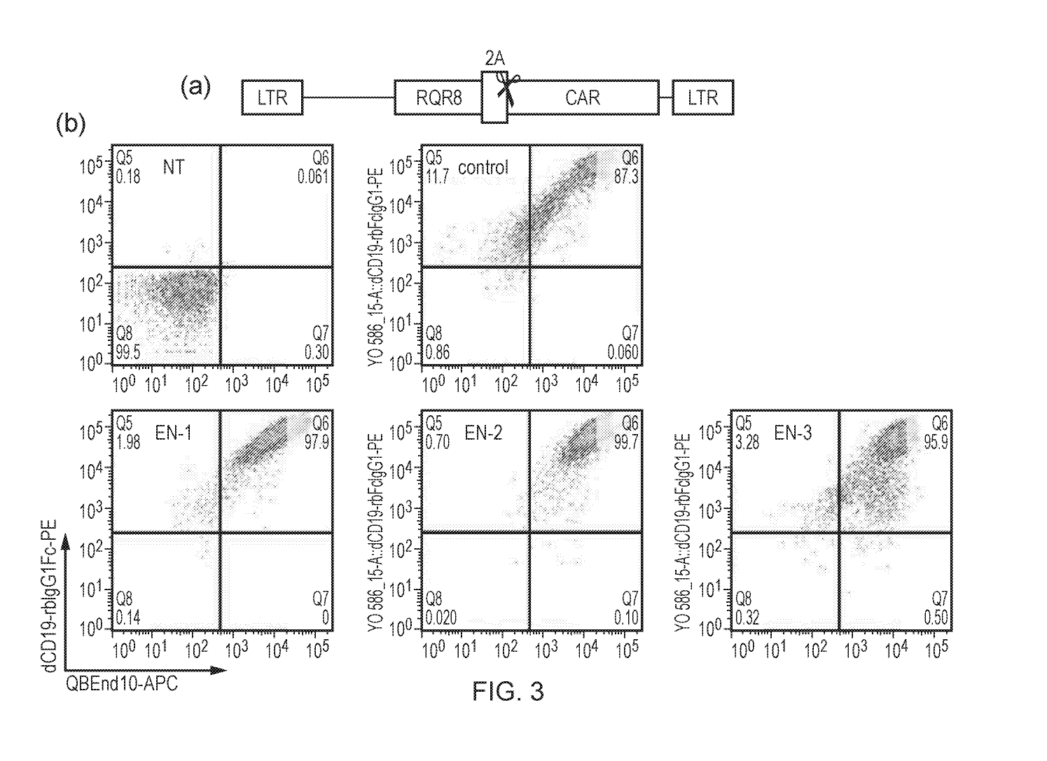

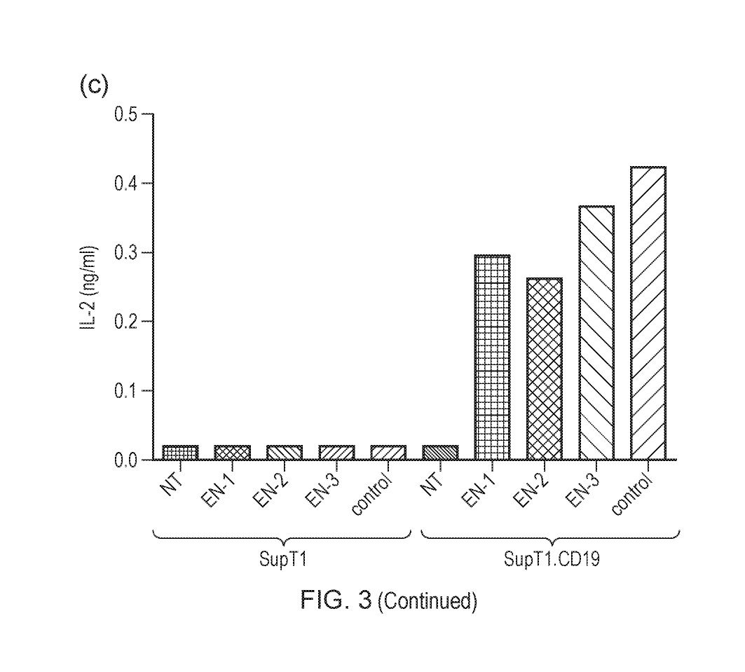

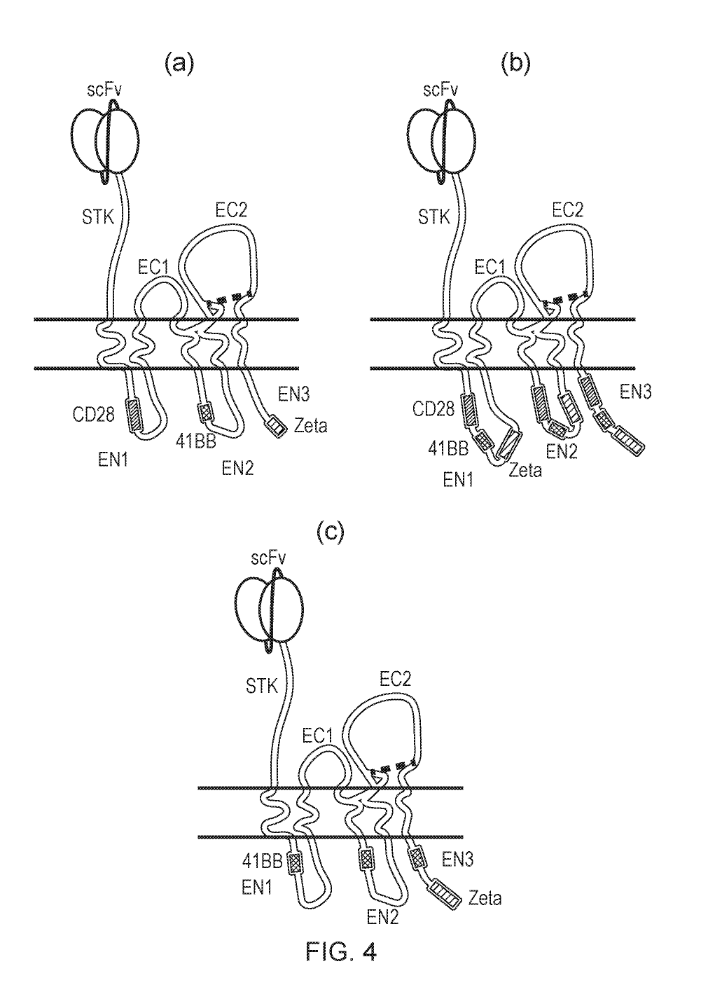

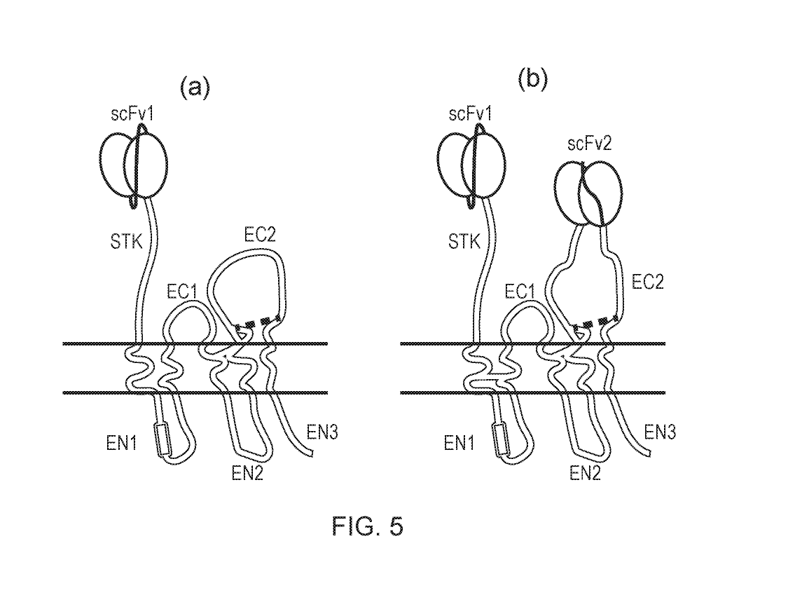

BW5 T-cells were transduced with these CARs or a classical 1st generation based on the same fmc63 scFv (control). Next, expression of the scFv was tested to determine if the multi-span protein can accept attachment of a scFv (FIG. 3). All the constructs were able to stably express the scFv as determined by bright fluorescence after staining with CD19-Fc fusion. Next, the T-cells we co-cultured with SupT1s target cells which are usually negative for CD19, and SupT1 cells engineered to express CD19. All CARs triggered equal amounts of IL-2 demonstrating that the signaling domains could be attached to all endodomains of a multi-span protein (see FIG. 3 (c)).

### Example 2

### Generation of Effective Multi-Span CARs Using a Range of Multi-Span Transmembrane Proteins and Inserting Signalling Domains into a Range of Intracellular Domains

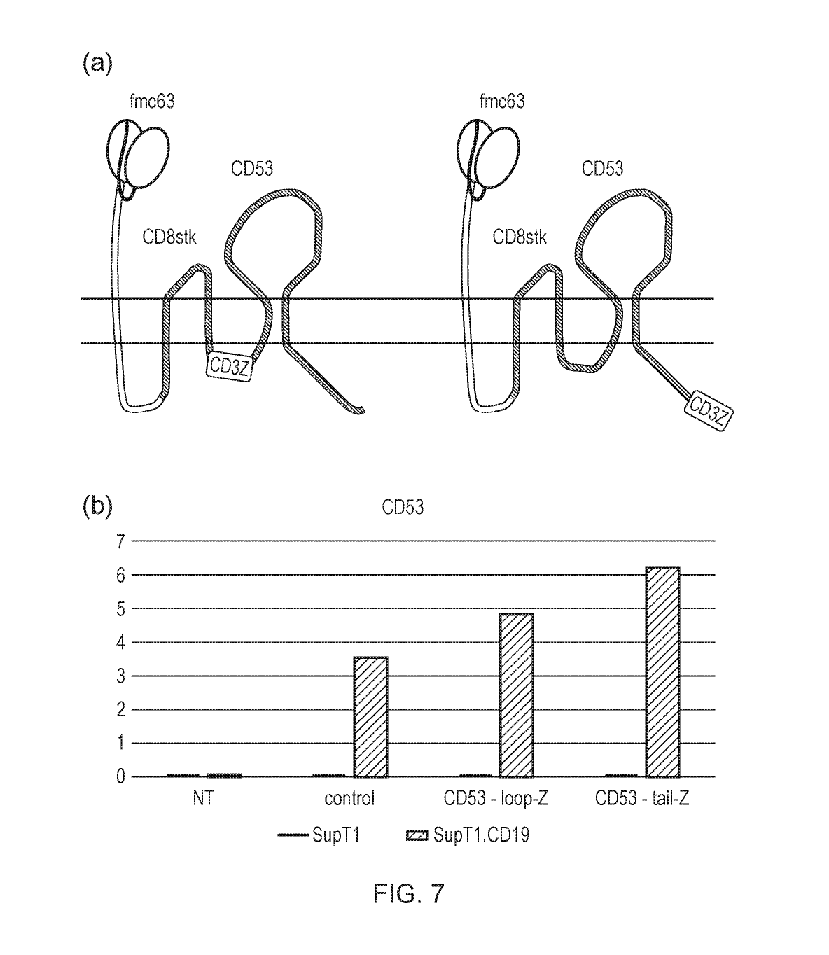

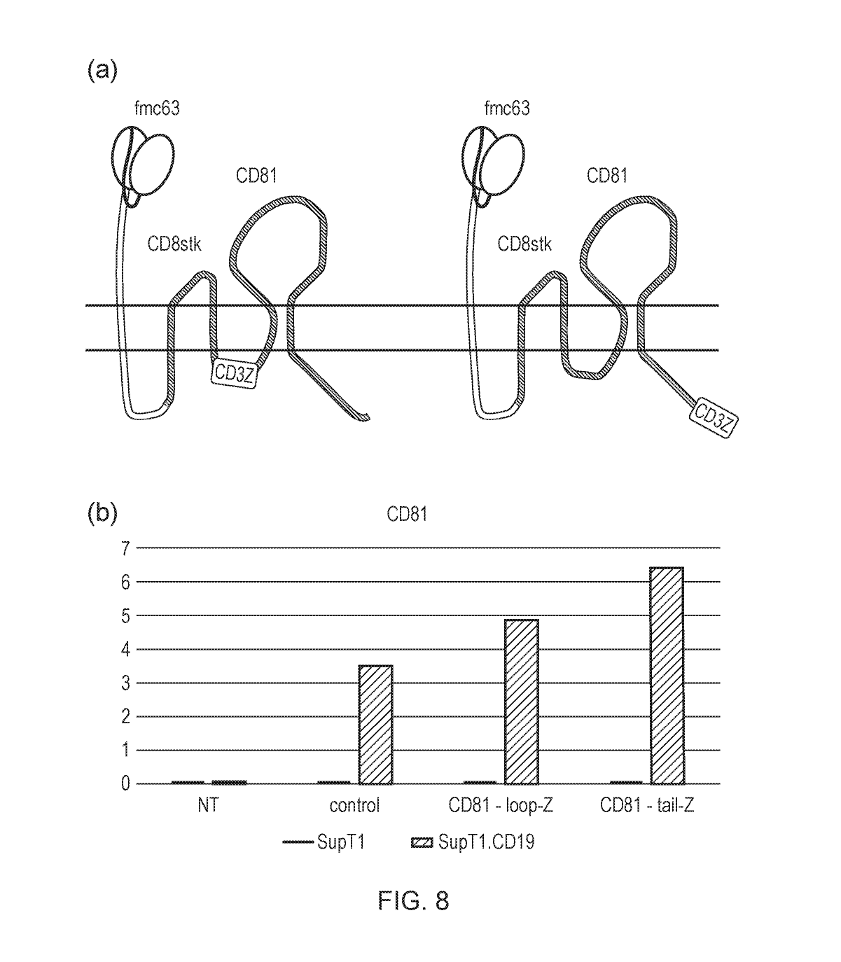

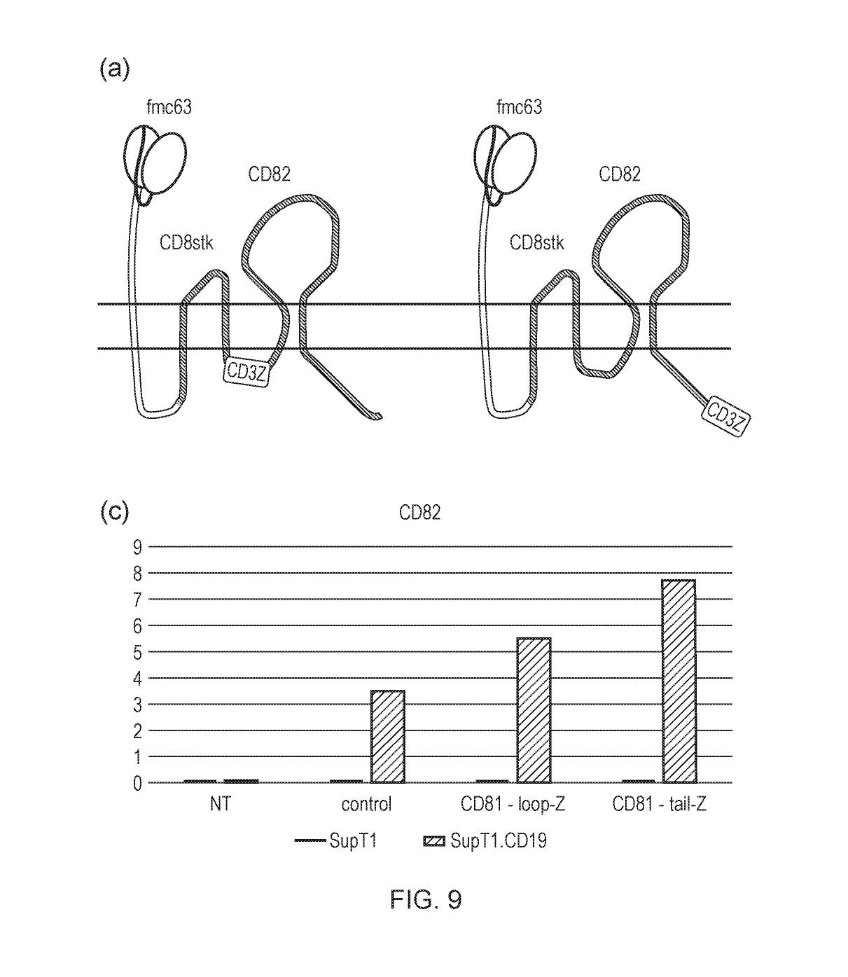

Several further multi-span transmembrane proteins other than CD20 were chosen and used to demonstrate that signalling from any intracellular loop or carboxy-termini of a multi-span transmembrane proteins is generally achievable. The proteins chosen were CD53, CD80 and CD81. The anti-CD19 scFv fmc63 attached to the CD8-stalk and transmembrane domain was attached to the amino-terminus of these proteins. Next, CD3-Zeta either replaced the carboxy-terminus or was inserted into the central intracellular loop. These different multi-span CARs were introduced into the T-cell line BW5s. A standard type I transmembrane protein fmc63 CD8-stalk CD3-Zeta CAR was used as a control. BW5 cells were challenged with SupT1 cells (which don't express CD19), and SupT1 cells engineered to express CD19. IL2 was measured from supernatant 24 hours after a 1:1 co-culture (FIGS. 7-9). All constructs triggered significant IL2 release specifically in response to CD19 expressing target cells indicating signalling. This demonstrates that multi-span CARs can be formed from different multi-span transmembrane proteins and signalling domains can generally be inserted into intracellular portions of multi-span transmembrane proteins and signal productively.

### Example 3

### Utility of Different Signalling Domains within a Multispan CAR

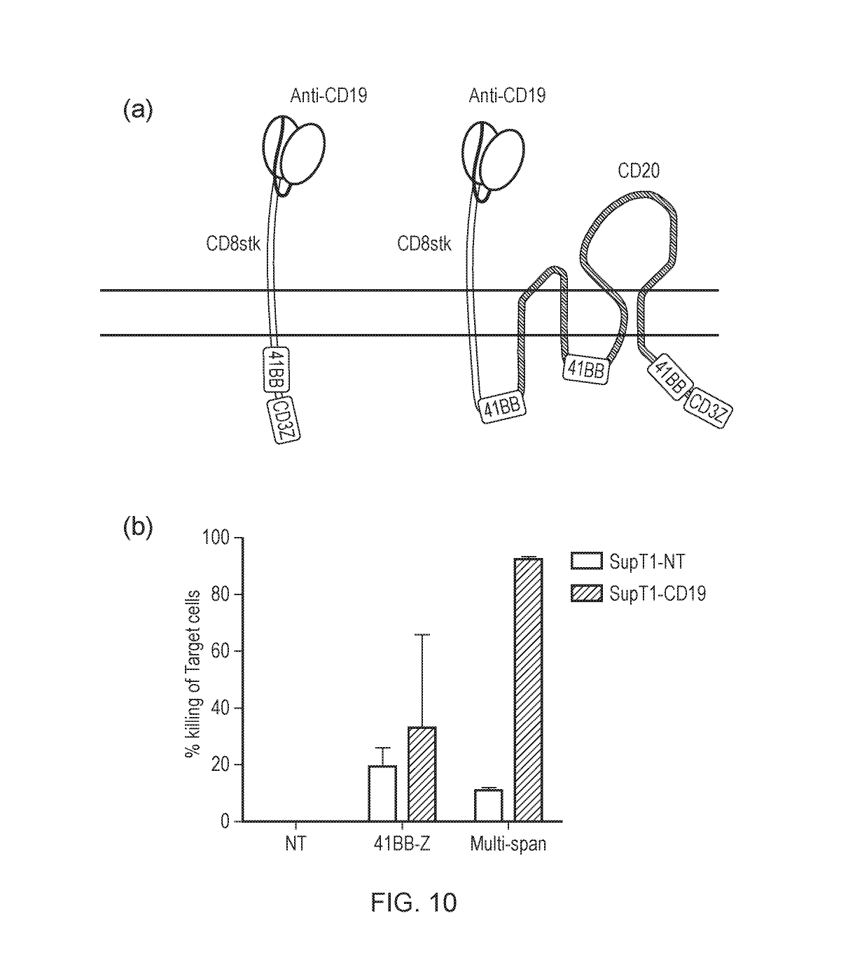

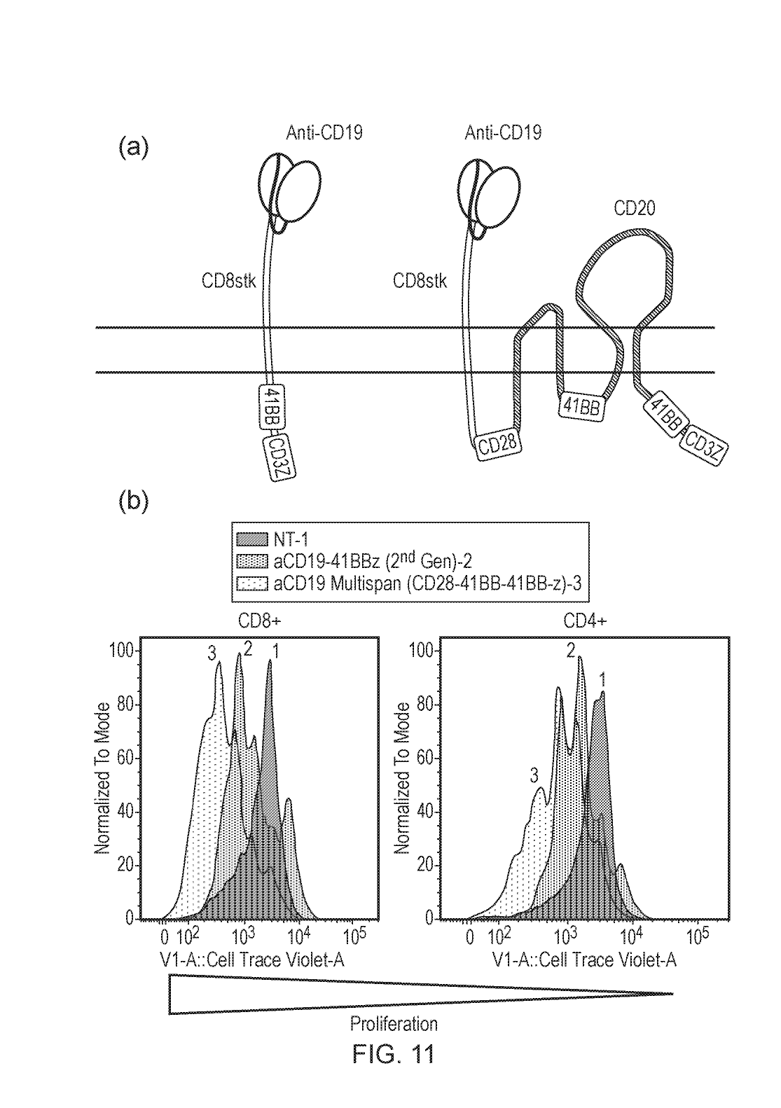

A multi-span CAR was generated by linking the anti-CD19 scFv fmc63 attached to the CD8-stalk and transmembrane domain to CD20 where the 41BB endodomain was inserted into the first and 2nd intracellular domains, and 41BB endodomain fused with the endodomain of CD3-Zeta replaced the third intracellular domain. Primary human T-cells were transduced with this construct. As a control, a standard CAR described by Campana (as above) composed of the fmc63 scFv attached to the CD8 stalk and transmembrane domain and 41BB endodomain fused with the endodomain of CD3-Zeta. T-cells were challenged with SupT1 cells (which normally do not express CD19), and SupT1 cells engineered to express CD19 under challenging conditions of 1 T-cell to 4 target cells for 24 hours. Multi-span CAR resulted in more effective killing of CD19+ targets than the standard CAR (FIG. 10). The multi-span CAR was modified so the first intracellular domain contained the endodomain of CD28 instead of that of 41BB. The endodomain of CD28 transmits a proliferative signal. T-cells were transduced to express this multispan CAR. This was compared with control T-cells expressing the standard Campana CAR as well as non-transduced T-cells. T-cells were labelled with CFSE and challenged with SupT1 cells engineered to express CD19 for 5 days. As the T-cells divide, CFSE gets diluted with each cell division. The multi-span CAR triggered more cell division than the standard CAR (FIG. 11).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A multi-span chimeric antigen receptor (CAR) which comprises:
i) at least one extracellular antigen binding domain;
ii) a plurality of linked transmembrane domains; and
iii) at least one intracellular signalling domain.

2. The multi-span CAR according to claim 1 which comprises more than one antigen-binding domain.

3. The multi-span CAR according to claim wherein each antigen binding domain is located at a different extracellular domain of the multi-span CAR.

4. The multi-span CAR according to claim 2, wherein each antigen binding domain recognises a different antigen.

5. The multi-span CAR according to claim 1 which comprises more than one intracellular signalling domain.

6. The multi-span CAR according to claim 5, wherein each intracellular signalling domain is located at a different intracellular domain of the multi-span CAR.

7. The multi-span CAR according to claim 5, wherein each intracellular signalling domain comprises a different signalling endodomain(s).

8. The multi-span CAR according to claim 5, wherein each intracellular signalling domain comprises the same signalling endodomain(s).

9. The multi-span CAR according to claim 1, wherein the intracellular signalling domain comprises at least one of CD3 zeta endodomain, CD28 endodomain, 41BB endodomain, OX40 endodomain, CD2 endodomain, Inducible T-cell costimulator (ICOS) endodomain, CD27 endodomain, BTLA endodomain, CD30 endodomain, GITR endodomain and HVEM endodomain.

10. The multi-span CAR according to claim 1 wherein the intracellular signalling domain comprises a single endodomain selected from CD3 zeta endodomain, CD28 endodomain, 41BB endodomain, OX40 endodomain, CD2 endodomain, Inducible T-cell costimulator (ICOS) endodomain, CD27 endodomain, BTLA endodomain, CD30 endodomain, GITR endodomain or HVEM endodomain.

11. The multi-span CAR according to claim 9, wherein the intracellular signalling domain comprises CD3 zeta endodomain, CD28 endodomain and 41BB endodomain or CD3 zeta endodomain, CD28 endodomain and OX40 endodomain.

12. The multi-span CAR according to claim 1 which comprises one or more of the transmembrane domains of CD20.

13. A nucleic acid encoding a multi-span CAR according to claim 1.

14. A vector comprising the nucleic acid sequence according to claim 13.

15. A cell which expresses the multi-span CAR according to claim 1.

16. The cell according to claim 15 which is a T cell or NK cell.

17. A pharmaceutical composition comprising the cell according to claim 15 and a pharmaceutically acceptable carrier, diluent or excipient.

18. (canceled)

19. A method for treating and/or preventing a disease which comprises the step of administering the pharmaceutical composition according to claim 17 to a subject.

20. A method for treating and/or preventing a disease, which comprises the following steps:
isolation of a cell containing sample from a subject;
(ii) transduction or transfection of the cell with a nucleic acid according to claim 13 or a vector comprising the nucleic acid; and
(iii) administering the cell from (ii) to the subject.

21. (canceled)

22. (canceled)

23. A method for making a cell which expresses a multi-span CAR, the method comprising introducing: a nucleic acid according to claim 13 or a vector comprising the nucleic acid into the cell.

24. The method according to claim 23, wherein the cell is from a sample isolated from a subject.
